(19) United States
(12) Patent Application Publication
(10) Pub. No.: US 2022/0273209 A1
(43) Pub. Date: Sep. 1, 2022

(54) DEVICES AND METHODS FOR SAMPLE COLLECTION

(71) Applicant: SISCAPA Assay Technologies, Inc., Washington, DC (US)

(72) Inventors: Norman Leigh ANDERSON, Washington, DC (US); Morteza RAZAVI, Victoria (CA)

(73) Assignee: SISCAPA Assay Technologies, Inc., Washington, DC (US)

(21) Appl. No.: 17/630,943

(22) PCT Filed: Jul. 28, 2020
(86) PCT No.: PCT/US20/43844

§ 371 Date: Jan. 28, 2022

Related U.S. Application Data
(60) Provisional application No. 62/879,621, filed on Jul. 29, 2019.
(60) Provisional application No. 62/954,530, filed on Dec. 29, 2019.
(60) Provisional application No. 62/958,834, filed on Jan. 9, 2020.
(60) Provisional application No. 63/006,992, filed on Apr. 8, 2020.
(60) Provisional application No. 63/035,232, filed on Jun. 5, 2020.

(51) Int. Cl. A61B 5/15; A61B 5/157

(52) U.S. Cl. CPC A61B 5/150305; A61B 5/150022; A61B 5/150358; A61B 5/150786; A61B 5/157

(57) ABSTRACT

Products and methods directed to, but not limited to, a sample collection device, a sample collection booklet and a sample collection kit for collection of blood or other bodily fluids, and analytical workflows, protocols, databases and other applications for collecting, sorting, classifying and saving and retrieving information on the subject and characterizations regarding the blood or other bodily fluids of the subject.

Incorporated by reference herein in their entirety are the contents of each of the below patent documents, each in its entirety:

U.S. Provisional Patent Application No. 63/035,232 filed 5 Jun. 2020

U.S. Provisional Patent Application No. 63/006,992 filed 8 Apr. 2020

U.S. Provisional Patent Application No. 62/958,834 filed 9 Jan. 2020,

U.S. Provisional Patent Application No. 62/954,530 filed 29 Dec. 2019,

U.S. Provisional Patent Application No. 62/879,621 filed 29 Jul. 2019,

U.S. Provisional Patent Application No. 62/651,523 filed Apr. 2, 2018,

U.S. Provisional Patent Application No. 62/626,843 filed Feb. 6, 2018,

U.S. Provisional Patent Application No. 62/618,486 filed Jan. 17, 2018

U.S. Provisional Patent Application No. 62/596,657 filed Dec. 8, 2017,

U.S. Provisional Patent Application No. 62/504,478 filed May 10, 2017,

U.S. Provisional Patent Application No. 62/418,538 filed Nov. 7, 2016,

U.S. Provisional Patent Application No. 62/362,443 filed Jul. 14, 2016,

U.S. Provisional Patent Application No. 62/343,705 filed May 31, 2016

U.S. Pat. No. 7,632,686 (application Ser. No. 10/676,005), entitled High Sensitivity Quantitation of Peptides by Mass Spectrometry; filed 2 Oct. 2003;

PCT/US2011/028569, entitled Improved Mass Spectrometric Assays For Peptides filed 15 Mar. 2011;

Ser. No. 11/256,946, entitled Process For Treatment Of Protein Samples, filed 25 Oct. 2005;

Ser. No. 12/042,931, entitled Magnetic Bead Trap and Mass Spectrometer Interface;

PCT/US 16/13876, entitled Combined Analysis Of Small Molecules And Proteins By Mass Spectrometry

PCT/US 13/48384—61/665,217—entitled Multipurpose Mass Spectrometric Assay Panels For Peptides

61/314,149, entitled MS Internal Standards at Clinical Levels filed on Mar. 15, 2010;

61/665,217, entitled Multipurpose Mass Spectrometric Assay Panels for Peptides filed on Jun. 27, 2012

60/415,499, entitled Monitor Peptide Enrichment Using Anti-Peptide Antibodies, filed 3 Oct. 2002;

60/420,613, entitled Optimization of Monitor Peptide Enrichment Using Anti-Peptide Antibodies, filed 23 Oct. 2002;

60/449,190, entitled High Sensitivity Quantitation of Peptides by Mass Spectrometry, filed 20 Feb. 2003;

60/496,037, entitled Improved Quantitation of Peptides by Mass Spectrometry, filed 18 Aug. 2003;

60/557,261, entitled Selection of Antibodies and Peptides for Peptide Enrichment, filed 29 Mar. 2004;

61/314,154 entitled Stable Isotope Labeled Peptides on Carriers, filed 15 Mar. 2010;

61/314,149 entitled MS Internal Standards at Clinical Levels filed 15 Mar. 2010;

61/665,217 entitled Multipurpose Mass Spectrometric Assay Panels For Peptides filed 27 Jun. 2012;

61/665,228 entitled Simultaneous Peptide And Metabolite Affinity Capture Mass Spectrometry filed 27 Jun. 2012;

61/670,493 entitled Proteolytic Digestion Kit With Dried Reagents filed 11 Jul. 2012;

61/720,386 entitled Peptide Fragments Of Human Protein C Inhibitor And Human Pigment Epithelium-Derived Factor And Use In Monitoring Of Prostate Cancer filed 30 Oct. 2012;

62/137,560 Devices For Collection Of Blood In Dried Form, filed 24 Mar. 2015;

The following documents are incorporated by reference herein in their entirety:

### REFERENCES

Each of the following references is incorporated herein in its entirety.
Altundemir, S.; Uguz, A. K.; Ulgen, K. A Review on Wax Printed Microfluidic Paper-Based Devices for International Health. Biomicrofluidics 2017, 11 (4), 041501. https://doi.org/10.1063/1.4991504.
Anderson N L, Anderson N G, Haines L R, Hardie D B, Pearson T W. Mass spectrometric quantitation of peptides and proteins using Stable Isotope Standards and Capture by Anti-Peptide Antibodies (SISCAPA). J Proteome Res. 2004 February; 3(2):235-44.
Anderson N L et al., “Precision multiparameter tracking of inflammation on timescales of hours to years using serial dried blood spots,” Bioanalysis, p. bio-2019-0278, April 2020, doi: 10.4155/bio-2019-0278.
Carrilho, E.; Martinez, A. W.; Whitesides, G. M. Understanding Wax Printing: A Simple Micropatterning Process for Paper-Based Microfluidics. Anal. Chem. 2009, 81 (16), 7091-7095. https://doi.org/10.1021/ac901071p.
Chambers A G, Percy A J, Yang J, Camenzind A G, Borchers C H. Multiplexed quantitation of endogenous proteins in dried blood spots by multiple reaction monitoring-mass spectrometry. 2013 March; 12(3):781-91.
E. D. Breen, J. G. Curley, D. E. Overcashier, C. C. Hsu, and S. J. Shire. Effect of Moisture on the Stability of a Lyophilized Humanized Monoclonal Antibody Formulation. Pharmaceutical Research, Vol. 18, No. 9, September 2001
Guthrie R, Susi A. A Simple Phenylalanine Method For Detecting Phenylketonuria In Large Populations Of Newborn Infants. Pediatrics. 1963; 32(3):338-43.
Lehmann S, Delaby C, Vialaret J, Ducos J, Hirtz C. Current and future use of “dried blood spot” analyses in clinical chemistry. Clinical Chemistry and Laboratory Medicine. 2013 October; 51(10):1897-909.
Lu, Y.; Shi, W.; Qin, J.; Lin, B. Fabrication and Characterization of Paper-Based Microfluidics Prepared in Nitrocellulose Membrane by Wax Printing. Anal. Chem. 2010,82 (1), 329-335. https://doi.org/10.102¹/ₐc9020193
R. H. Ellis, T. D. Hong, and E. H. Roberts, “A Comparison of the Low-Moisture-Content Limit to the Logarithmic Relation Between Seed Moisture and Longevity in Twelve Species,” Ann. Bot., vol. 63, no. 6, pp. 601-611, June 1989, doi: 10.1093/oxfordjournals.aob.a087788.
Razavi M, Leigh Anderson N, Pope M E, Yip R, Pearson T W. High precision quantification of human plasma proteins using the automated SISCAPA Immuno-MS workflow. New Biotechnology. 2016 Sep. 25; 33(5 Pt A):494-502.
Rosting C, Gjelstad A, Halvorsen T G. Water-Soluble Dried Blood Spot in Protein Analysis: A Proof-of-Concept Study. Anal Chem. 2015 Aug. 4; 87(15):7918-24.
Statland B E, Bokelund H, Winkel P. Factors contributing to intra-individual variation of serum constituents: 4. Effects of posture and tourniquet application on variation of serum constituents in healthy subjects. Clin Chem. 1974; 20:1513-9.
Tanna S, Lawson G. Self-sampling and quantitative analysis of DBS: can it shift the balance in over-burdened healthcare systems? Bioanalysis. 2015 September; 7(16):1963-6.
H. Wang et al., “Direct Analysis of Biological Tissue by Paper Spray Mass Spectrometry,” Anal. Chem., vol. 83, no. 4, pp. 1197-1201, February 2011, doi: 10.1021/ac103150a.

Dried blood spot cards and devices of various types including:

Whatman 903 & DMPK cards made by GE Healthcare Life Sciences (and described at the website https://www.cytivalifesciences.com/en/us/shop/whatman-laboratory-filtration/whatman-dx-components/blood-collection-cards-and-accessories/903-proteinsaver-snap-apart-card-p-00677#overview).

AutoCollect made by Ahlstrom (and described at the website https://www.ahlstrom-munksjo.com/products/medical-life-sciences-and-laboratory/specimen-collection-cards/automated-microvolume-samping-cards/).

Mitra made by Neoteryx (and described at the website www.neoteryx.com, and in patent documents EP 2 785 859, 2017/0023446, 2017/0043346, 2017/0128934, US2013/0116597).

hemaPEN made by Trajan Scientific and Medical (and described at the website http://www.trajanscimed.com/pages/hemapen, and in WO002017024360A1).

HemoLink made by Tasso, Inc. (and described at the website www.tassoinc.com, and in US2013/0211289 and US2014/0038306).

Capitainer made by Capitainer (and described at the website www.Capitainer.se).

Hemaxis made by DBS System SA (and described at the website http://hemaxis.com/services/).

TAP100 Touch Activated Phlebotomy made by 7th Sense Bio (and described at the website http://www.7sbio.com/about/, and in).

HemaSpot HF made by Spotonsciences (and described at the website http://www.spotonsciences.com/).

PTS PodTM Blood Collection System made by PTS Diagnostics (and described at the website http://www.ptsdiagnostics.com/pts-pod-system.html).

Noviplex Plasma Prep Card made by Novilytic Laboratories (and described at the website https://novilytic.com/).

Advance Dx 100 plasma collection card made by Advance Dx, Inc. (and described at the website http://www.adx100.com/more_info.htm).

ViveBio plasma separation card made by ViveBio LLC (and described at the website http://www.vivebio.com/scientific_literature.html).

Asante Dried Blood Specimen Collection Strip made by Sedia Biosciences (and described at the website http://www.sediabio.com/products/blood-specimen-collection-devices).

Fluispotter made by Fluisense (and described at the website http://www.fluisense.com/).

Descriptions of the ViveST device contained in U.S. Pat. Nos. 7,638,099; 8,334,097; and 8,685,748; U.S. Design Pat. No. D631,169, and U.S. application Ser. Nos. 14/020,142 and 14/165,877

Descriptions of the “Mitra” absorber material in U.S. Pat. No. 7,638,099 and US20130116597

Calibrated capillary micropipettes (e.g., Drummond Scientific)

Matrix 2D Barcoded Storage Tubes (https://www.matrixtechcorp.com/storage-systems/tubes.aspx?id=63) and Fluidx 96-Well Format Sample Storage Tubes with Screw Cap and 2D Barcode (http://www.fluidx.eu/96-well-format-sample-storage-tubes-with-2d-barcode.html)

Various commercially available absorbable gelatin or collagen sponges such as SURGIFOAM® Absorbable Gelatin Sponges by Ethicon and GELFOAM Sterile Compressed Sponge made by Pfizer.

Formed zeolite tablets as described in U.S. Pat. No. 4,214,011.

Incorporated by reference herein in their entirety are the contents of each of the below patent documents:

| Category | Patent or Application |
|---|---|
| GenTegra | US20100209957 |
| GenTegra | US2014/0295429 |
| Hopfgartner | EP2484448A1 |
| Miscellaneous | CN203483432U |
| Miscellaneous | CN205002991U |
| Miscellaneous | CN205181357U |
| Miscellaneous | US 2003/0156996 |
| Miscellaneous | US 2013/0071944 |
| Miscellaneous | U.S. Pat. No. 6,274,304 |
| Miscellaneous | U.S. Pat. No. 6,582,290 |
| Miscellaneous | U.S. Pat. No. 7,638,099 |
| Miscellaneous | U.S. Pat. No. 8,685,748 |
| Miscellaneous | US20030178328 |
| Miscellaneous | US20030195435 |
| Miscellaneous | US20040265187 |
| Miscellaneous | US20050227269 |
| Miscellaneous | US20070208275 |
| Miscellaneous | US20130116597 |
| Miscellaneous | U.S. Pat. No. 4,214,011 |
| Miscellaneous | U.S. Pat. No. 5,520,041 |
| Miscellaneous | U.S. Pat. No. 5,665,238 |
| Miscellaneous | U.S. Pat. No. 5,747,265 |
| Miscellaneous | U.S. Pat. No. 6,036,659 |
| Miscellaneous | U.S. Pat. No. 6,106,732 |
| Miscellaneous | U.S. Pat. No. 6,524,533 |
| Miscellaneous | US20130211289_1 |
| Miscellaneous | US20140038306_2 |
| Neoteryx | EP 2 785 859 |
| Neoteryx | US 2017/0023446 |
| Neoteryx | US 2017/0043346 |
| Neoteryx | US 2017/0128934 |
| Neoteryx | US2013/0116597 |
| Seventh Sense Biosystems, Inc. | EP3243435A1 |
| Seventh Sense Biosystems, Inc. | JP2015211878A |
| Seventh Sense Biosystems, Inc. | US20100069726A1 |
| Seventh Sense Biosystems, Inc. | US20100249560A1 |
| Seventh Sense Biosystems, Inc. | US20100269837A1 |
| Seventh Sense Biosystems, Inc. | US20100272652A1 |
| Seventh Sense Biosystems, Inc. | US20100330703A1 |
| Seventh Sense Biosystems, Inc. | US20110009847A1 |
| Seventh Sense Biosystems, Inc. | US20110105872A1 |
| Seventh Sense Biosystems, Inc. | US20110105951A1 |
| Seventh Sense Biosystems, Inc. | US20110105952A1 |
| Seventh Sense Biosystems, Inc. | US20110172508A1 |
| Seventh Sense Biosystems, Inc. | US20110172510A1 |
| Seventh Sense Biosystems, Inc. | US20110181410A1 |
| Seventh Sense Biosystems, Inc. | US20120010529A1 |
| Seventh Sense Biosystems, Inc. | US20120016308A1 |
| Seventh Sense Biosystems, Inc. | US20120039809A1 |
| Seventh Sense Biosystems, Inc. | US20120041338A1 |
| Seventh Sense Biosystems, Inc. | US20120123297A1 |
| Seventh Sense Biosystems, Inc. | US20120271125A1 |
| Seventh Sense Biosystems, Inc. | US20120275955A1 |
| Seventh Sense Biosystems, Inc. | US20120277696A1 |
| Seventh Sense Biosystems, Inc. | US20120277697A1 |
| Seventh Sense Biosystems, Inc. | US20130158468A1 |
| Seventh Sense Biosystems, Inc. | US20130158482A1 |
| Seventh Sense Biosystems, Inc. | WO2011065972A2 |
| Seventh Sense Biosystems, Inc. | WO2016123282A1 |
| Trajan | WO002017024360 |
| Vivebio | US20140038172 |
| Vivebio | US20140141411 |
| Vivebio | U.S. Pat. No. 7,638,099 |
| Vivebio | USD631169 |
| Weighing blood for donation | US20040186408A1 |
| Weighing blood for donation | US20090081785A1 |
| Weighing blood for donation | U.S. Pat. No. 3,924,700 |
| Weighing blood for donation | U.S. Pat. No. 4,294,320 |
| Weighing blood for donation | U.S. Pat. No. 4,449,538 |
| Weighing blood for donation | U.S. Pat. No. 4,922,922 |
| Weighing blood for donation | U.S. Pat. No. 4,923,449 |
| Weighing blood for donation | U.S. Pat. No. 5,064,541 |
| Weighing blood for donation | U.S. Pat. No. 6,524,533 |
| Capitainer | US10220389 |

### FIELD AND BACKGROUND

This invention relates to quantitative assays for evaluation of proteins and other analytes in complex samples such as human blood, urine, sputum, ascites, synovial fluid and cerebrospinal fluid, and specifically to the collection, transport, storage and preparation of samples for such assays.

There is a need for improvement in the collection and processing of liquid human samples, including whole blood, plasma, serum, urine, saliva and cerebrospinal fluid, for the measurement of proteins, metabolites, drugs, and nucleic acids for a variety of purposes including research and clinical diagnostics. Blood is a primary clinical specimen, and represents the largest and deepest version of the human proteome present in any sample: in addition to the classical “plasma proteins” and the cells of red cells, white cells and platelets, it contains all tissue proteins (as leakage markers) plus very numerous distinct immunoglobulin sequences; and it has an extraordinary dynamic range, in that more than 10 orders of magnitude in concentration separate albumin and the rarest proteins now measured clinically. Abundant scientific evidence, from proteomics and other disciplines, suggests that among these are proteins whose abundances and structures change in ways indicative of many, if not most, human diseases. Nevertheless, only about 100 proteins are currently used in routine clinical diagnosis, while the rate of introduction of new protein tests approved by the US FDA has paradoxically declined over the last decade to about one new protein diagnostic marker approved per year. Furthermore, it appears that the clinical value of most such tests would be substantially improved if the results were interpreted in terms of patient-specific (i.e., personalized) baselines (rather than population reference intervals)—an advance that is currently inhibited by the cost and inconvenience of collecting a series of baseline samples from each patient before the emergence of major disease processes. Major advances in diagnostics are to be expected if certain technical problems in sample collection, preparation and analysis are solved. This specification focuses on issues of the collection and preparation of suitable samples from blood, although the disclosed processes can be used for other sample types as well.

Human blood, and the serum and plasma samples derived from it, is typically collected and prepared in evacuated glass or plastic tubes (known colloquially as “Vacutainers”). In the usual course of medical practice, these tubes are filled by venipuncture and sent to a clinical laboratory for analysis, where they may be stored for extended periods (hours to days) at room temperature or 4 C. It would be useful to obtain small samples of blood by skin prick instead of venipuncture, thus allowing collection of blood samples for protein measurement by a patient at home, and to stabilize such samples in order to facilitate transport to an analytical laboratory.

Drying is one such method of stabilization applicable to blood. Based on Guthrie's implementation (Guthrie, 1963)) of dried blood spots (DBS) on filter paper for newborn screening, dried samples have been investigated in a variety of contexts for a decade or more. Numerous publications have confirmed that a wide array of metabolites, drugs and proteins can be measured in such samples (Lehmann, 2013; Chambers, 2013) and that individuals can perform effective finger prick sample collection at home (Tanna, 2015). DBS samples are not fully equivalent to conventional venipuncture specimens in terms of accurately known plasma volume or concentration of some biomarkers (e.g., proteins elevated in interstitial fluid compared to venous blood), but these limitations can be largely overcome using new MS-based analytical methods.

Determining blood analyte concentrations from dried samples is complicated by the fact that concentrations of large analytes (such as proteins or circulating cells) can change significantly due to shifts in the distribution of water between the blood and other tissues. Albumin and total protein concentrations in blood can change by 5-10% over 30 min depending on posture, whereas small analytes like sodium and potassium are hardly affected (Statland, 1974). In order to reduce this variation in samples acquired under field conditions (where control of patient posture before sample collection may not be rigorously controlled) it would be useful to be able to measure the amount of water in the blood sample in relation to the non-aqueous solutes and solids (cells, proteins, lipids, ions, etc.).

Mass spectrometric assays using DBS samples are especially attractive because by digesting the proteins to peptides, generally with trypsin or other proteolytic enzymes, and then measuring surrogate peptides that are unique to each protein (“proteotypic peptides”) by mass spectrometry (MS), the problem of protein stability over time is alleviated. From the MS viewpoint, this approach has the effect of transforming the protein measurement problem into a small molecule quantitation problem, where isotope dilution methods are effective and well understood.

The basic components for conventional dried blood spot collection are a lancet to pierce the skin and a paper blood collection card. Following a finger prick using a disposable lancet applied to a finger cleaned with an alcohol swab, the user attempts to apply blood to a collection card, for example a Whatman 903 card, typically attempting to place one drop of blood on each of the 5 preprinted circles on the card. After drying in air for at least 2 hours, the card may be folded closed and stored in a desiccated bag, ideally at 4 C or −20 C. Placement of the blood drops is difficult for many individuals as they must squeeze, or “milk” the punctured finger in order to extract sufficient blood while simultaneously steering the forming droplet (which is difficult to see since it hangs beneath the lanced finger) into a circle without actually touching the finger to the paper. The volumes of the drops produced and the size to which they spread is not well controlled, leading to variation in the amount of blood in a punch taken from the dried card for analysis. In addition, the components of clotting blood (plasma, cells and coagulum) may be differentially transported in the paper as the blood drop spreads out, leading to differences in composition at different points in the dried drop, further complicating measurement of true blood concentrations of biomarkers.

The conventional method of using DBS samples is to punch a small circle from a blood-containing region of the paper, typically about ¼ inch in diameter. Differences in amount of blood retained by that area of paper, in blood hematocrit (which affects viscosity and hence spreading), in coagulation and chromatography during drop spreading and in preferential drying near the edge of the blood-soaked region, all result in variations away from the bulk composition of a homogeneous applied blood sample. It would therefore be preferable to analyze a sample that represents the entirety of a volume of collected blood, rather than a potentially variable subset.

While dried blood spot cards can be barcoded and otherwise labeled effectively, when a punch is removed from the card, the identification of the sample must be transferred to vessel into which the punch is placed without error. It would therefore be preferable to avoid the movement of the sample out of its original identified format during processing, and in particular to avoid movement of the sample in any form that is not somehow labeled in an error-free manner.

To facilitate determination of analyte concentrations in terms of mass per volume of blood (or its serum, plasma or cellular constituents), it can be useful to collect and stabilize a pre-determined volume of blood, and to analyze all of this volume rather than a region of a potentially inhomogeneous spot.

In order to test for low-abundance biomarkers, it would also be beneficial to analyze samples larger than conventional ¼″ blood spot punches, which contain on average only about 14 ul of blood and 7 ul of plasma. While a single dried blood spot typically represents one drop, or about 25 ul, of blood, it is difficult to cut out the whole spot and introduce it into a vessel for extraction since the whole spot has a larger diameter than the diameter of a standard 96-well plate well (typically 6-8 mm).

While the conventional DBS collection procedure relies on drying the sample in ambient air, which can vary in humidity and temperature over a wide range, it would also be useful to provide means of drying the sample quickly and reproducibly to a very low humidity independent of ambient conditions.

For those analytical procedures that require digestion of the proteins to peptides (e.g., by exposure to a proteolytic enzyme such as trypsin), it would be useful to provide a means for executing this digestion conveniently and reproducibly on samples without the need to divide or transfer the sample to a secondary container.

For longitudinal studies, in which serial samples are collected from one individual over time, it would be useful to have a compact device and associated complete ample collection kit specifically designed for this purpose and capable of collecting and preserving a series of samples, rather than a handful of separate devices used one after another. Likewise, it would be preferable to provide a device and method for rapid collection of samples, rather than a time-consuming process that disrupts other scheduled patient activities.

### SUMMARY

The present invention addresses these problems by providing means for collecting small blood samples and stabilizing by drying in a device that facilitates automated processing of the entire sample once the sample arrives at the analytical laboratory. Reagents, such as synthetic stable-isotope labeled peptides used as internal standards for quantitation, can be incorporated into the sample collection device as well.

The invention is equally applicable to protein samples from sources other than blood, such as tissue homogenates, small tissue pieces, animal, plant or microbial samples, other body fluids, environmental samples and the like. While the device and methods are described mainly in terms of sample collection for protein analysis, other biomolecules, such as DNA and RNA, drugs or metabolites, as well as non-biological environmental chemicals can likewise be collected, processed and stabilized.

A general approach for protein biomarker quantitation involves digesting proteins (e.g., with trypsin) into peptides that can be further fragmented (MS/MS) in a mass spectrometer to generate a sequence-based identification, lending specificity to the MS quantitative measurement. The approach can be used with either electrospray (ESI) or MALDI ionization, and is typically applied after one or more dimensions of chromatographic or affinity (e.g., SISCAPA) fractionation to reduce the complexity of peptides introduced into the MS at any given instant. Preparation of peptides from a sample such as plasma is typically carried out by first denaturing an aqueous protein sample (e.g., with detergents such as deoxycholate, organic solvents, urea or guanidine HCl), reducing the disulfide bonds in the proteins (e.g., with tris(2-carboxyethyl)phosphine (TCEP), dithiothreitol or mercaptoethanol), alkylating the cysteines (e.g., by addition of iodoacetamide, or iodoacetic acid, which reacts with the free —SH group of cysteine), quenching excess iodoacetamide by addition of more dithiothreitol or mercaptoethanol, and finally (after removal or dilution of the denaturant) addition of the selected proteolytic enzyme (e.g. trypsin), followed by incubation to allow digestion. Following incubation, the action of trypsin can be terminated, either by addition of a chemical inhibitor (e.g., TLCK) or by denaturation (through heat or addition of denaturants, or both) or removal (if the trypsin is on a solid support) of the trypsin.

SISCAPA assays (Anderson, 2004; Razavi 2016; Anderson, 2020) combine affinity enrichment of specific peptides from such a sample digest with quantitative measurement of those peptides by mass spectrometry. In order to detect and quantitatively measure protein analytes, the SISCAPA technology makes use of anti-peptide antibodies (or any other binding entity that can reversibly bind a specific peptide sequence of about 5-20 residues) to capture specific peptides from a mixture of peptides, such as that arising, for example, from the specific cleavage of a protein mixture (like human serum or a tissue lysate) by a proteolytic enzyme such as trypsin or a chemical reagent such as cyanogen bromide. By capturing a specific peptide through binding to an antibody (the antibody being typically coupled to a solid support either before or after peptide binding), followed by washing of the antibody:peptide complex to remove unbound peptides, and finally elution of the bound peptide into a small volume (typically achieved by an acid solution such as 5% acetic acid), the SISCAPA technology makes it possible to enrich specific peptides that may be present at low concentrations in the whole digest, and therefore undetectable in simple mass spectrometry (MS) or liquid chromatography-MS (LC/MS) systems against the background of more abundant peptides present in the mixture. It also provides a sample that is less complex, and therefore exhibits lesser ‘matrix effects’ and fewer analytical interferences, than observed in the starting digest. This in turn permits mass spectrometry analysis without further separation steps, although additional separation processes could be used if desired. The sample can be concentrated prior to analysis if necessary, but this concentration does not provide any further analyte peptide separation. This enrichment step is intended to capture peptides of high, medium or low abundance and present them for MS analysis: it therefore discards information as to the relative abundance of a peptide in the starting mixture in order to boost detection sensitivity. This abundance information, which is of great value in the fields of proteomics and diagnostics, can be recovered, however, through the use of isotope dilution methods: the SISCAPA technology makes use of such methods (e.g., by using stable isotope labeled versions of target peptides) in combination with specific peptide enrichment, to provide a method for quantitative analysis of peptides, including low-abundance peptides. The approach to standardization in SISCAPA is to create a version of the peptide to be measured which incorporates one or more stable isotopes of mass different from the predominant natural isotope, thus forming a labeled peptide variant that is chemically identical (or nearly-identical) to the natural peptide present in the mixture, but which is nevertheless distinguishable by a mass spectrometer because of its altered peptide mass due to the isotopic label(s). In one embodiment, the method for creating the labeled peptide is chemical synthesis, wherein a peptide with chemical structure identical to the natural analyte can be made while incorporating amino acid precursors that contain heavy isotopes of hydrogen, carbon, oxygen or nitrogen (e.g., 3H, 13C, 18O or 15N) to introduce the isotopic label. In theory one could also use radioactive (i.e., unstable) isotopes (such as 3H), but this is less attractive for safety reasons. The isotopic peptide variant (a Stable Isotope-labeled Standard, or SIS) is used as an internal standard and is added to the sample peptide mixture at a known concentration before enrichment by antibody capture. The antibody captures and enriches both the natural and the labeled peptide together (having no differential affinity for either) according to their relative abundances in the sample. Since the labeled peptide is added at a known concentration, the ratio between the amounts of the natural and labeled forms detected by the final MS analysis allows the concentration of the natural peptide in the sample mixture to be calculated. Thus, the SISCAPA technology makes it possible to measure the quantity of a peptide of low abundance in a complex mixture, and since the peptide is typically produced by quantitative (complete) cleavage of sample proteins, the abundance of the parent protein in the mixture of proteins can be deduced. The SISCAPA technology can be multiplexed to cover multiple peptides measured in parallel, and can be automated through computer control to afford a general system for protein measurement. Creating a new protein-specific assay thus, requires only that a peptide-specific antibody and a labeled peptide analog be created. A feature of the SISCAPA technology is directed at establishing quantitative assays for specific proteins selected a priori, rather than at the problem of comparing all of the unknown components of two or more samples to one another. It is this focus on specific assays that makes it attractive to generate specific antibodies to each monitor peptide (in principle one antibody binding one peptide for each assay).

The SISCAPA method, including prior sample digestion, has been fully automated using conventional robotic liquid handling systems acting on samples in 96 well plates (7). The introduction of dried blood spot samples into such 96 well plates remains however only partially automated (e.g., using the PerkinElmer Panthera-Puncher 9) which punches small circular regions of the DBS card (typically ¼″ diameter) into designated wells guided by an operator holding the card. Variations in the blood content of the punched region, or its composition relative to the applied blood, result in analytical error.

In the descriptions that follow, quantitation of proteins, peptides and other biomolecules is addressed in a general sense, and hence the invention disclosed is in no way limited to the analysis of blood, plasma and other body fluids. The instant invention uses several of the cited methods of the prior art in an entirely different combination.

The present invention relates to devices and methods for collecting, stabilizing and further processing biological samples including blood. The invention allows a volume of blood to be introduced into a container, absorber or substrate, and dried by a desiccant in a closed space isolated from the variable humidity of the external atmosphere. The invention allows collection and processing of multiple samples, either at once or at different times. The invention provides for verification of the amount of sample collected by means of weight measurements on a dried sample and/or sample water extracted by a weighed desiccant. The invention allows collected samples to be directly interfaced with laboratory robotic sample handling technology without manual intervention. Samples collected according to the invention are identified by unique machine-readable codes to establish a chain of custody from collection to analysis. Sample collection according to the invention can be carried out while making use of mobile computing devices (e.g., smartphones) capable of assisting the user and adding important information (e.g., GPS location) to a sample-associated record transmitted to and stored in a remote repository (e.g., in the cloud).

### BRIEF DESCRIPTION OF THE DRAWINGS

FIGS. 1A-1C. Device for collection of a multiple samples on a patterned card of absorber material.

FIGS. 2A-2B. Arrangement of impermeable barrier zones on a patterned card.

FIG. 3. A composite sampling device with absorbent tines connected to an inert body

FIG. 4. A composite sampling device with multiple pre-placed reagent zones, and method of sequential dissolution by addition of liquid

FIGS. 5A-5B. Placement of the sampling device into a 96-well plate for processing.

FIGS. 6A-6D. Views of a sample collection book including four 8-tine pages (collection devices), bound with separator sheets.

FIGS. 7A-7F. Components of a sample collection book.

FIG. 8. Photograph of a sample collection book with 4 8-tine collection devices, sample information sheet and other components in a small ring binder.

FIG. 9. Application of blood samples to the device of FIGS. 6A-6D.

FIG. 10. Sample collection device with dried blood samples placed in a numbered slot in a fan-fold storage system.

FIG. 11. Sample collection kit including sample collection booklet, lancet, desiccant in mesh bag and RH/Temp logger in airtight plastic box.

FIG. 12. Record of relative humidity (RH) inside a sample collection kit recorded over a month

FIG. 13. Photograph of an 8-tine sample collection device with blood applied to 6 of the 8 sample tines

FIG. 14. Montage of cellphone photographs of 8 consecutive samples collected in a sample collection device also referred to as a comb device.

FIG. 15. Diagram of a sample collection card in a closed package with desiccant tablets and sealed but openable capillary channels for application of sample to individual sample zones.

FIGS. 16A-16C. Photographs of a sample collection card in a closed package with desiccant tablets and sealed but openable capillary channels before and after application of sample to individual sample zones.

FIGS. 17A-17B. 8-tine sampling device curled within cylindrical or trumpet-shaped housings such that one sample application tine is accessible and others are shielded.

### DETAILED DESCRIPTION

The invention provides devices and associated methods for collecting and stabilizing biological samples for identification and quantitative analysis of peptides and/or proteins, metabolites, drugs, DNA and RNA therein. While many of the devices and methods known in the art and disclosed above are useful with the methods of the invention, the specification for such a commercially useful process has not previously been disclosed.

The terms “analyte”, and “ligand” may be any of a variety of different molecules, or components, pieces, fragments or sections of different molecules that one desires to measure or quantitate in a sample. The term “monitor fragment” may mean any piece of an analyte up to and including the whole analyte, which can be produced by a reproducible fragmentation process (or without a fragmentation if the monitor fragment is the whole analyte) and whose abundance or concentration can be used as a surrogate for the abundance or concentration of the analyte. The term “monitor peptide” means a peptide chosen as a monitor fragment of a protein or peptide.

The term “sample” refers to a quantity of material to be collected for later analysis, and includes liquid samples such blood, plasma, serum, cerebrospinal fluid, synovial fluid, lymph, tears, saliva, bronchial-alveolar lavage fluid, nasal swab fluid, blister fluid, urine, vaginal fluid and phlegm. Samples can also include small pieces of solid material capable of adhering to a collection device, such as small tissue biopsies, skin samples, and pathology specimens.

The term “biomolecules” refers to any molecule present in a biological system, and includes proteins, nucleic acids (specifically DNA and RNA in its various forms, both intracellular and extracellular), complex sugars (glycans and the like), lipids, and a variety of metabolites.

The term “capillary” refers to a material component having an internal cavity with internal wall surfaces sufficiently hydrophilic and cross-sectional dimensions sufficiently small so as to cause aqueous solutions (including blood) to be drawn into the cavity by capillary forces. In its simplest form a capillary may be a tube made of glass, but the term also includes non-cylindrical forms (e.g., gaps between opposing flat surfaces), as well as water- and/or analyte-permeable materials such as paper, or various polymers.

The terms “proteolytic treatment” or “enzyme” may refer any of a large number of different enzymes, including trypsin, chymotrypsin, lys-C, v8 and the like, as well as chemicals, such as cyanogen bromide. In this context, a proteolytic treatment acts to cleave peptide bonds in a protein or peptide in a sequence-specific manner, generating a collection of peptide fragments referred to as a digest.

The term “denaturant” includes a range of chaotropic and other chemical agents that act to disrupt or loosen the 3-D structure of proteins and other complex molecules without breaking covalent bonds, thereby rendering them more susceptible to proteolytic treatment, more soluble, or both. Examples include chaotropes such as urea, guanidine hydrochloride, ammonium thiocyanate; detergents such as sodium dodecyl sulfate, cetyltrimethyl ammonium bromide, Triton X-100; as well as solvents such as acetonitrile, ethanol, methanol and the like.

The term “desiccant” means a material capable of binding water and removing it from the air, so as to lower humidity, or directly from a contacting liquid. Desiccants include silica gel, calcium chloride, activated alumina, and most important in the present context, zeolite molecular sieve such as 3 A or 4 A having a very high capacity to tightly bind water while not absorbing larger molecules. Preferred desiccant materials are zeolite molecular sieves, for example 3 A, whose approximate chemical formula is given by 2/3K₂O.1/3Na₂O.Al₂O₃.2SiO₂.9/2H₂O.

The term “bind” includes any physical attachment or close association, which may be permanent or temporary. Generally, reversible binding includes aspects of charge interactions, hydrogen bonding, hydrophobic forces, van der Waals forces, etc., that facilitate physical attachment between the molecule of interest and the analyte being measured. The “binding” interaction may be brief as in the situation where binding causes a chemical reaction to occur. Reactions resulting from contact between the binding agent and the analyte are also within the definition of binding for the purposes of the present invention, provided they can be later reversed.

The terms “internal standard”, “isotope-labeled monitor fragment”, or “isotope-labeled monitor peptide” may be any altered version of the respective monitor fragment or monitor peptide that is 1) recognized as equivalent to the monitor fragment or monitor peptide by the appropriate binding agent and 2) differs from it in a manner that can be distinguished by a mass spectrometer, either through direct measurement of molecular mass or through mass measurement of fragments (e.g., through MS/MS analysis), or by another equivalent means.

“SIS” or “stable isotope standard” means a peptide, or protein containing a peptide, having a unique sequence derived from the protein product of a gene and including a label of some kind (e.g., a stable isotope) that allows its use as an internal standard for quantitation (see U.S. patent application Ser. No. 10/676,005 “High Sensitivity Quantitation of Peptides by Mass Spectrometry”). Included peptides may have non-material modifications of this sequence, such as a single amino acid substitution (as may occur in natural genetic polymorphisms), substitutions outside the region of contact (including covalent conjugations of cysteine or other specific residues), or chemical modifications to the peptide (including glycosylation, phosphorylation, and other well-known post-translational modifications) that do not materially affect binding.

The terms “absorbent material”, “support”, “absorber”, “substrate”, “imbiber” or “imbibition zone” include any porous or absorbent material in membrane, sheet, tubular, bead, plug, particulate or other forms whose structure defines an included volume, and which can imbibe a liquid sample by capillary action or surface tension. Examples include papers (for example Whatman 903 and Ahlstrom 226 papers), porous polymeric materials as described in U.S. Pat. No. 7,638,099 and US20130116597, open-cell foams, or solid substrates patterned so as to generate numerous holes, channels or grooves having hydrophilic surfaces. A support can comprise one or more porous materials embedded or dispersed within other porous materials. A support can also be composed of particles embedded within another porous material (e.g., 3M Empore® membranes). A support can also be a material that maintains its shape during sample imbibition and drying, but which can be disassembled to yield a homogeneous suspension of sample plus suspended absorber particles or fibers (AQUACEL® Ag BURN Hydrofiber® Dressing can, for example, be used as such a soluble absorber (Rosting, 2015)), or the absorber can be a dissolvable sponge material such as an absorbable collagen or gelatin sponge (e.g., SURGIFOAM® Absorbable Gelatin Sponges by Ethicon or GELFOAM Sterile Compressed Sponge made by Pfizer) which can be rendered completely soluble during the process of sample digestion (e.g., through the action of trypsin). The material of the support, and particularly the surface (internal and external) exposed to an imbibed liquid, is referred to as the matrix.

The term “imbibition” means the absorption of liquid into a porous support by means of capillary forces not requiring hydrostatic pressure, and applies to supports that swell as well as those that do not.

The term “imbibe” is meant to describe the process whereby a liquid is drawn into a porous material by forces of capillary action or surface tension. When a liquid sample is fully imbibed into a support, it is fully contained within the support, leaving minimal residual liquid outside the volume described by the outer surface of the support. The process of imbibition into a homogeneous support zone ensures that all elements of the liquid are exposed equally to enzymes or reagents evenly distributed within the support zone.

The term “paper” as used herein means any porous material in the form a sheet or strip. This includes conventional cellulosic papers, non-cellulosic membranes made of plastics (PVDF, polycarbonate, etc.), and membranes that are or are not homogeneous through their thickness (i.e., including plasma separation filter membranes). It also includes sheet materials formed as in paper manufacturing, or through other industrial processes such as those involving precipitation, spraying, extrusion, stretching, molding, drawing, rolling, etc. Such sheet materials may be of constant thickness, or may vary in thickness.

The term “comb” is used herein to describe a relatively flat shape having a “body” from which project a series of “tines”; i.e., similar in general shape to a common hair comb in which a series of parallel tines are connected at one end to a rigid body. Each tine is thus a strip of material joined at one end to a body, parallel to one or more other tines that are also joined to the body, typically with uniform spacing between tines. Such a comb is generally of planar form, although when formed of a flexible material, such a comb can be bent into non-planar shapes. A comb and can be made by cutting the comb from planar material, by molding, by additive manufacturing or other processes.

The term “drug” is used herein to refer to a chemical compound or biomolecule administered to a human or animal for pharmaceutical purposes, including treatment of disease.

The term “sugar” as used herein means any water-soluble (or sample soluble) solute that when dried in a paper or membrane can render the paper of membrane water or sample impermeable. The term thus includes many types of sugars, salts, polymers and other molecules that can be used to render a paper material temporarily water-impermeable in order to better define the internal volume of a paper-sampling device.

The terms “wax”, varnish”, “silicone”, “water-repellent” or “hydrophobic” as used herein refer to a variety of known chemicals, formulations, treatments and coatings that are used to alter the surface character of materials, including paper, so as to reduce the propensity for water or aqueous solutions to wet the material, whether applied to surfaces or as fillings that permeate the material.

The terms “patterning” or “printing” refer to the placement of a coating, including hydrophobic coatings, on a planar absorber such as paper or a paper-like material so as to define a pattern of hydrophobic areas, leaving the remainder of the surface with different properties (e.g., hydrophilic).

The term “barcode” as used herein refers to any of a wide variety of computer-readable or scanner-readable image representations of numbers and/or text, including linear barcodes of various type, 2D barcodes (including common QR codes), and computer-readable Optical Character Recognition (OCR) fonts, and the like. As used here, barcodes can serve to identify unique devices (e.g., sample collection devices) by direct scanning of the device itself, or by recognition of and interpretation of the barcode in a photograph of the device (for example a photograph taken upon collection of a sample using the device). It is common practice, followed here, to include a human-readable version of the identifier along with a barcode so as to allow a person to read the identifier with or without the aid of a scanning device or computer.

Description of the Device

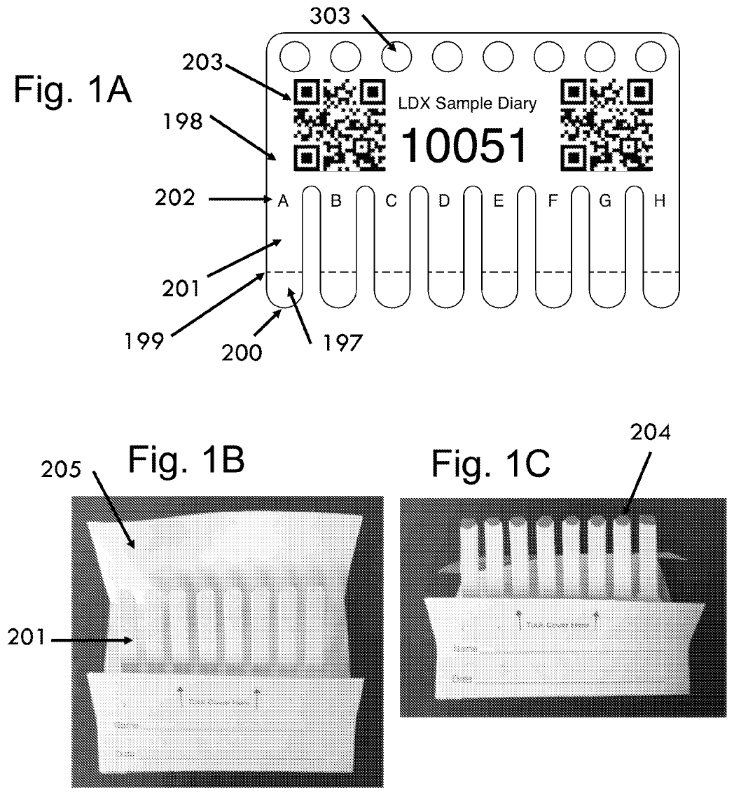

In its simplest form, the device of the invention is comprised of a body region that connects two or more projecting regions (“tines”), together comprising a “comb” device. A proximal end of each tine is connected to the body, and a distal end, or “tip”, of each tine projects away from the body. The tip of a tine provides a sample application zone or sample application point sufficiently distant from the body and from other tines' application zones or points that a droplet of sample placed into contact with one tine's sample application zone or point does not contact other tines' sample application zones or points, or the body. The distal end of a tine adjacent to, and in some embodiments including, the sample application zone or point is formed of a material capable of imbibing a volume of a liquid sample, and thus comprises a sample region or sample zone. The material forming the sample zone may have a number of other particular properties including chemical purity, absence of substances that could alter or contaminate a sample, consistent physical behavior, and origin in a process governed by an approved quality system to enable use in regulated medical or other applications. The sample application zone or point may comprise an edge of the device material, providing a site where a droplet of liquid sample contacts a very limited surface area of imbibing material, thus restricting the speed of liquid uptake and aiding in control of filling the sample zone. A sample region is entirely confined to one tine and does not extend into the body, thus preventing contact of one sample with any other sample. The device is uniquely identified by one or more markings, which may include computer-readable codes, such as barcodes or QR codes, or by human-readable codes including written alphanumeric codes or symbols of non-alphanumeric writing systems. These unique markings can be applied to and identify each tine of the device (thus identifying each sample applied to the device by its tine's unique markings) or unique markings can be applied to the body of the device, while the individual tines within the device are uniquely identified by a secondary marking on each tine in a device (e.g. A-H markings in FIG. 1A) or by virtue of each tine's relative position relative to the body. A liquid sample applied to the sample application zone or point of a tine and subsequently imbibed in the sample zone of the tine and dried in place provides a positionally fixed specimen that can be physically manipulated without directly contacting the dried sample by movement of the body of the comb device. A set of distinct samples applied to different tines of a device can be placed into a series of separate respective vessels, for example a column of wells in a standard 96-well plate, by a single manipulation of the device. A specimen dried on a tine of the device can be subdivided by removal of one or more portions of the sample region without destroying the physical integrity of the remainder of the comb and the samples or portions thereof remaining in the comb, for example by slicing off the tips or ends of one or more tines. The body or tines of the device can comprise attachment sites such as holes or handles that facilitate manipulation of the device while minimizing unintended contact or contamination, or facilitate combination with other similar devices or additional components to produce compound objects with additional features or functions.

A preferred embodiment of a sample collection device according to the invention can be formed from a sheet of absorbent paper (e.g., Ahstrom 226 paper, Whatman 903 paper, or another absorbent material suitable for collection of biological samples) shaped as shown in FIGS. 1A-1C. The device, described here and referred to below as a sample collection comb, sample collection device or sampling device, comprises a body region 198 and a series of projecting parallel tines 201 forming sample application zone ends.

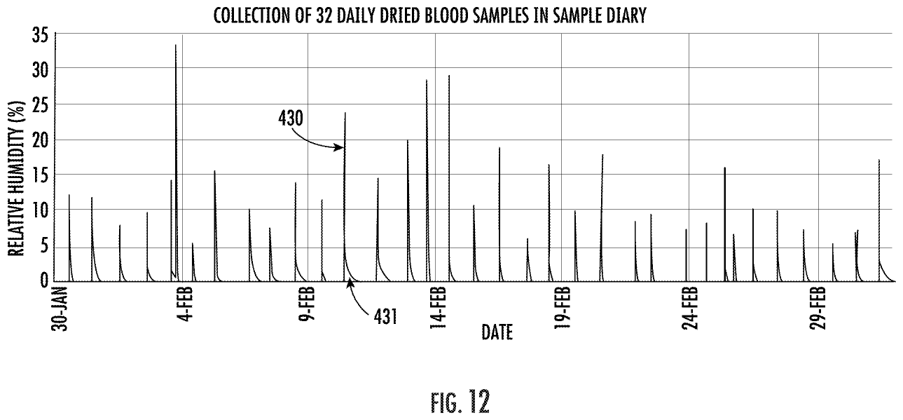

The tine projections may be parallel in the sense of having uniformly shaped projections and/or uniform spacing between tines as shown in the figures or may be parallel in the sense of having uniformly spaced longitudinal center lines to permit placement in standard uniformly spaced multiwell plates, but where the tines and the spaces between the tines need not be uniformly shaped. In a preferred embodiment, the device has 8 (as in FIGS. 1A-1C) or 12 tines, and the spacing between the longitudinal center lines of the tines is equal to the spacing between wells in a standard 96-well plate format used in laboratory automation (i.e., 9 mm), allowing insertion of the comb tines into a column (8 tines) or row (12 tines) of wells in a plate 96-well plate having 8 rows of 12 columns of wells in a rectangular array. In another preferred embodiment, the device has 16 or 24 tines, and the spacing between the longitudinal center lines of the tines is equal to the spacing between wells in a standard 384-well plate format used in laboratory automation (i.e., 4.5 mm), allowing insertion of the comb tines into a column (16 tines) or row (24 tines) of wells in a plate 384-well plate having 16 rows of 24 columns of wells in a rectangular array. Additional embodiments with spacing between tines equal to the well-to-well spacing of other devices comprising multiple sample wells will be apparent to those skilled in the art. In an embodiment, the zones defined herein contain or imbibe uniform volumes per area on all the tines to permit standardization of sample and reagent content on the tines.

In the embodiment shown in FIG. 1A, the tip, or sample application zone end, 200 of a tine provides a region in which sample can be applied to the tine, for example by contacting a droplet of sample with tip 200, but without contacting other tines or the body of the comb. The distance between the tips of adjacent tines in this embodiment (9 mm spacing) provides ample space to allow contact of a tip 200 with a droplet of a sample such as blood without risk of accidently touching another tip. Spacings larger than 4 mm between adjacent tips are preferred for applications in which humans place samples on the collection device manually, while smaller spacings can be used for applications in which samples are placed by mechanical devices.

The sample application zone ends at the tips 200 of the tines comprise edges of the absorbent material as well as upper and lower planar surfaces. Such an edge, particularly the edge at the tip 200 of a tine, is capable of imbibing liquid sample in an easily controlled manner, for example by contacting a liquid droplet of sample with the edge of the material from which contact point the sample is continuously wicked into the material, progressively filling an increasing area of the sample zone 197. During the filling of the sample zone by wicking into this edge, which may last for a number of seconds, the user is able to terminate or restart sample inflow easily by simply moving the sample droplet out of, or back into, contact with the edge. Sample loading by contact of a droplet with the planar surface of the material, e.g., in the middle of sample zone 197, occurs much more quickly because of the large area of contact, and is therefore more difficult to control.

In the embodiment shown in FIGS. 1A-1C, the tips are rounded so as to accommodate the shape of so-called “round-bottom” 96-well plates, but the tips can have other contours including rectangular (so as to accommodate the shape of flat-bottom 96-well plates) or pointed (so as to accommodate the shape of “V”-bottom 96-well plates, or the requirements of a spray tip as used in paper-spray mass spectrometry).

A design such as that shown can be cut from sheets of the paper using a steel rule die, a laser, or a variety of paper cutting, converting or processing technologies known in the art. Alternatively, the device can be formed in a mold from a porous material such as that used in Neoteryx VAMS tips, or constructed by additive manufacturing methods such as 3D printing by powder sintering well known in the art.

In a preferred embodiment, the body of the comb has a series of holes 303 to facilitate manipulation and handling, including combination with other sheets in a ring- or spiral-bound book as described below. These holes can be cut from the paper using a steel rule die, a laser, or a variety of paper processing means known in the art, or molded into the device. In the embodiment shown in FIG. 1A, the holes are spaced so as to match the spacing of rings in a multi-ring binder such as Swingline GBC Proclick Binding Spines, and the holes are large enough and placed so as to allow free movement of the device when bound in such a binding. The number of holes can be reduced and still function with a binding having fewer rings, for example to just the two nearest the right and left sides of the device.

The device is identified by unique human-readable and computer-readable labels (in this case a QR code 203 encoding the same text as the human-readable label: “LDX Sample Diary 10051”). These labels are preferably unique to each device, and may be generated in a serial order during the manufacturing process to simplify tracking of the collected samples. The individual tines can be identified by tine identifiers 202, e.g., letters (e.g., A through H) or numbers (e.g., 1-8) to distinguish the individual samples collected on the different tines and for convenience of users in selecting the correct tine on which a sample is to be applied. Together, the device QR code and the tine identifier provide a unique identification of each individual sample collected on a tine (e.g., the first sample on the left-most tine is “LDX Sample Diary 10051A”, and the sample on the second tine is “LDX Sample Diary 10051B”, etc. . . . Alternatively, or in addition, each tine can have a unique barcode to identify it (e.g., a linear barcode printed in a vertical orientation along the tine) in the event that the tines are separated at some stage during collection or analysis. A design comprising human and computer readable information such as that shown can be printed on paper using a using a variety of printing means known in the art.

A line 199 can be printed, embossed, scored or perforated at a distance from the tip of each tine to indicate the limit of the area that should be filled by imbibition of sample applied at the tip 200. In the case of blood, it is easy for the user to observed the imbibition of red blood from the tip upwards towards line 199, and thus the user can cease inflow once that line is reached. For clear samples such as serum, CSF, urine, etc., inclusion of an inert dye in the paper can be used to make the inflow of sample visible and therefore controllable by the user.

In a preferred embodiment, line 199 is perforated, presenting a slight barrier to flow of the sample towards the body of the device. This partial restriction of sample flow ensures that sample fully fills the region from tip 200 to the line 199 before continuing to flow towards the body of the device. The ability of any excess applied sample to continue flowing past the perforated line ensures that the paper in the intended sample region (between tip 200 and line 199) is not overfilled when drying, thus helping standardize the amount of sample collected and subsequently dried in the sample area. For some applications a plurality of lines can be used indicating multiple target sample volumes.

The comb device can be made of a wide variety of materials capable of imbibing a liquid sample (e.g., a blood, serum, plasma, urine, CSF or synovial fluid sample) or carrying a small solid sample (e.g., a needle biopsy, aspirate, tissue fragment, skin biopsy or the like). Such materials include cellulosic papers, synthetic polymer papers, sintered polymers (such as the material used in Neoteryx devices). An extremely wide range of paper-like materials have been developed for printing, filtration, mechanical, and artistic applications, and many of these may be used in the devices described here. The material of the comb can be formed from sheets by mechanical cutting, laser cutting, etc. The stiffness of the planar material of the device can be improved by corrugated it, so as to in one or both dimensions, or it can be reinforced with infused stiffeners, lacquers, or other applied materials, etc. Many methods are known in the art for coating planar materials to alter physical properties including stiffness, strength, etc.

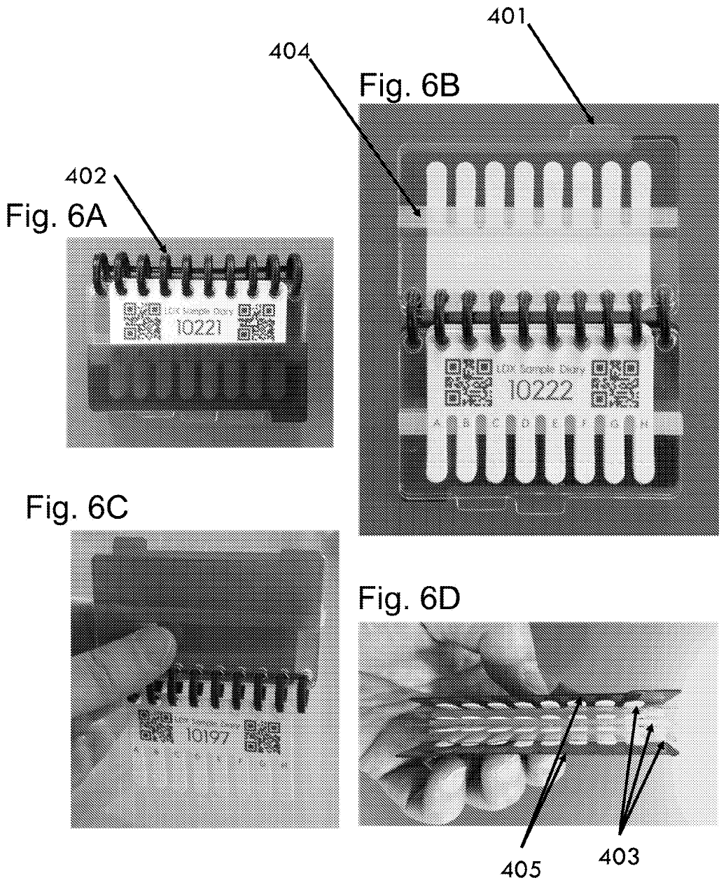

The preferred embodiment shown in FIGS. 1 and 6 is made of Ahlstrom 226 paper and the sample application region below the perforated line 199 takes up a volume of approximately 15 uL of a liquid sample such as blood. In another preferred embodiment, the perforated (or printed) fill line can be place higher up the tine (towards the body of the device) to provide a larger sample application region of 20, 30, 40, 50, or 60 uL, or any desired volume that can be contained in the paper of a suitably shaped tine without extending into the body of the device and mixing with adjacent samples. In another preferred embodiment, the device is made from thicker paper, allowing the collection of a larger sample volume. For example, use of paper twice the thickness of Ahlstrom 226 paper results in uptake of twice as much sample between the tip and the perforated or printed fill line as would be taken up by the single layer device. Alternatively, a laminated paper composition made by laminating 2 sheets of 226 paper together using adhesive, glue, sewing, crimping or other means known in the art for sticking together sheets of material, can be used to produce a similar increase in the volume of sample taken up by each tine of the device. In another preferred embodiment, two sheets of 226 paper can be laminated with a rigid inert material between them to give approximately double the sample capacity of the single layer device. In each of these embodiments directed at increasing the amount of sample that can be absorbed into the device, a variety of materials besides paper can be used, including porous or sintered polymers in sheets or molded into the form of the invention.

The comb device can also be made of materials capable of separating plasma from whole blood, generally by filtering out the blood cells and platelets in a filtration material. Such materials are commonly used in lateral flow diagnostic devices to prepare plasma from whole blood for analysis, and are available in formats where the separation takes place during flow through a membrane (i.e., flow normal to the plane of the membrane), or flow along (i.e., within the plane of) the membrane. In a preferred plasma-generating embodiment, a comb device is created in which the tines are composed of a material capable of separating plasma from blood cells by flow within the plane of the material. When a blood sample is applied to the tip of a tine of the comb sample collection device, plasma flows (is wicked) through the material along the tine towards the body of the device as a result of capillary forces. This creates a zone of plasma separated from, and adjacent to, a zone of blood cells that is retained on the tip of the tine, the plasma zone being “inboard” of the blood cells, closer to the body of the device. After the separation has taken place, the samples can be dried as for the previously described devices, or they can be used while still wet for direct analysis. In such a device, the zones of blood cells and plasma are visible, and can be separately cut from the sample device for separate analysis. For example, the tips of the tines can be cut off and placed in vessels for analysis of blood cell contents; and intermediate zone containing both blood cells and plasma can be preferably cut away and discarded, and finally a zone of purified plasma cut from the stubs of the tines for analysis of plasma constituents. Alternatively small regions (e.g., circles) can be punched or cut out of the tine at any position from the tip to the body of the device in order to access “pure” plasma or blood cell fractions, and the locations of these punches can be prespecified or they can be determined by computerized analysis of an image of the tine in which regions of pure fractions and the boundary between them can be identified.

In one embodiment (FIG. 1B) the individual device can be enclosed in a protective carrier, e.g., a protective paper wrapper 205 similar to the wrapper provided in conventional dried blood spot cards. In use, liquid blood samples are applied to the ends of the tines (FIG. 1C), resulting in blood-filled zones 204. This blood dries to form dried blood samples which are stable for transport and storage.

Number of Tines

A series of alternative designs will be obvious to those skilled in the art . The comb could carry 12 tines instead of 8, compatible with filling a 96 well plate by rows instead of columns. Combs could be made with finer spacing to place samples in 384 well plates. Combs of any number of tines can be made according to the invention, with any spacing required to match an analytical workflow requirement.

In a further embodiment, a roll of sample collection material (e.g., paper) can be cut so as to remain continuous while having a continuous series of tines projecting from one side: i.e., the material is cut to form a continuous comb. The material can be printed so as to provide a unique identification to each tine. An unlimited series of samples can be applied to individual tines by scrolling along such a roll. The samples can be dried immediately after loading and before the applied sample area is rolled up, or the collection material can be rolled up with spacers between successive layers in the roll side to prevent the collection material in one layer from touch the next, and allowing sample water to migrate our though the resulting space. At any point, the roll can be cut, e.g., into lengths of 8 or 12 tines to form sample collection devices similar to that shown in FIG. 1A, suitable for placement in a standard 96-well plate format. Such a roll-format device can collect any number of samples, and providing advantages for field collection of large sample sets in a compact package. In such an embodiment, with spacing between tines aligned so that each tine of the sample collection device will place into a single well of a multi-well plate for robotic automated processing, for example, wells that are spaced by 9 mm (well center to well center) according to standard laboratory formatting and e.g., spacing between tines aligned so that each tine of the sample collection device will individually fit into single well of a multi-well plate for robotic automated processing. For example, the longitudinal centers of the tines are uniformly spaced, for example at 9 mm from center to center, thus permitting 1000 tines(1,000 samples) to occupy a spool of 9 meters length, with a diameter less than 8 inches.

Barrier

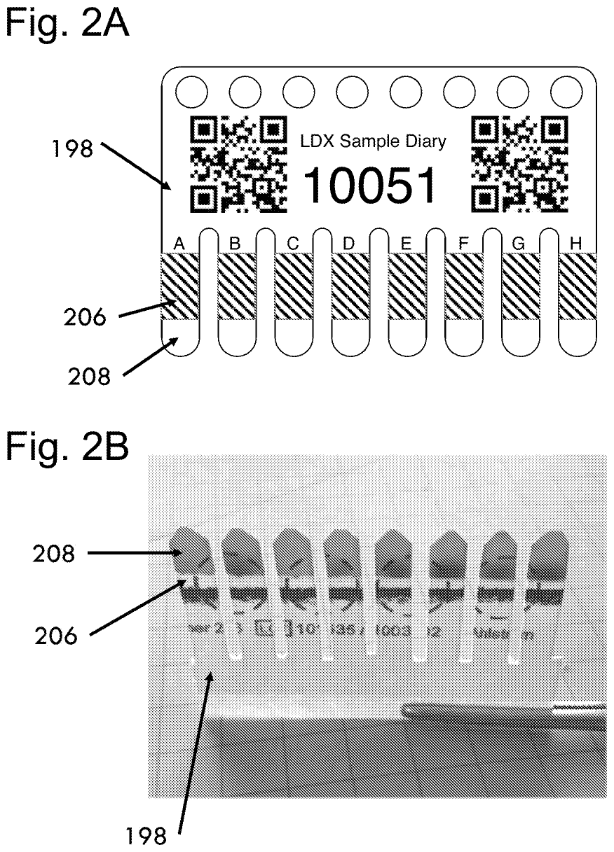

The amount of blood taken up by the tines can be determined approximately by creating a liquid impermeable barrier or barrier zone, e.g., a blood-impermeable barrier 206 across the tines, as shown in FIG. 2A, such that e.g., blood applied at the tip can be imbibed by the sample application zone 208 up to the barrier 206 but will not flow beyond. Such barriers can be created by applying wax, hydrophobic oils, paints, glues or the like in a line across the tines, or by crushing the paper to impeded flow. In various configurations the barrier can be a narrow line at a position such as 199 (FIG. 1A), or can extend most or all of the length of the tine(s) above the sample zone e.g., zone 206 in FIG. 2A). In a further embodiment the entire device, except for the sample application zone(s) 208, is treated as a barrier and coated with waxes, hydrophobic oils, paints, or glues, etc. Such a coating of the device apart from the sample areas may additionally enhance the stiffness, rigidity, and strength of the device, thus contributing to improved performance and resistance to damage in handling.

A significant feature of embodiments including an impermeable barrier such as 206 in FIG. 2A is that only the sample containing region 208 is permeable to aqueous reagents used in analysis of the sample. For example, when a device such as shown in FIG. 1A, loaded with samples dried in regions 208, is inserted into a 96-well plate containing aqueous buffers used to dissolve the sample for analysis, the aqueous buffer can be imbibed by the paper of the tines and transported upwards, potentially filling the body of the comb, resulting in the loss of the liquid in the well and the sample it contains. When the device shown in FIG. 2A is inserted into a 96-well plate with aqueous solvent, the sample contained in 208 can be dissolved, but the solvent will not enter the zone 206 due to its lack of capability to imbibe aqueous solvents, and thus the bulk of the sample (lacking only the volume still included in the paper of region 208) remains in the well and available for further processing in an analytical workflow.

In the example shown in FIG. 2B, a very thin layer of wax was applied to front and back surfaces of Ahlstrom 226 paper (except for the tips of the comb tines 208 where blood was subsequently loaded; in this illustration the tines point upward instead of downward as in other figures) by heating the paper between two sheets of common kitchen waxed paper before sample was applied. In this device, the distal edge of the wax-coated zone 206 stopped inflow of blood 208 at a sharp line, indicating that the surface wax effectively prevented inflow of blood into the paper where the surface was waxed. Such control of liquid flow is consistent with published literature on control of liquid flow in papers and membranes by wax printing (Altundemir, 2017; Carrilho, 2009; Lu, 2010).

Composite Construction

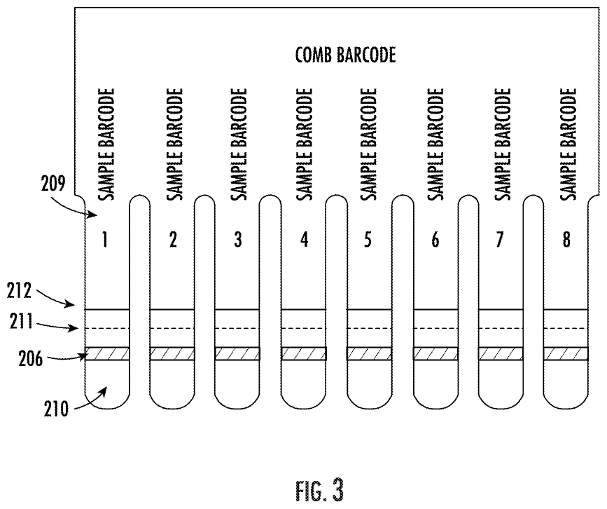

FIG. 3 shows an alternative composite embodiment of the device in which the sample zones 210 of the tines are made of sample absorbing material (extending from the tip of the tine to 212), while the body of the comb 209 is made of a planar semi-rigid preferably inert material such as plastic (e.g., polypropylene), extending to 211. The sample application zones or tips 210 located at the end of the tines are attached to the body 209 through interaction in the overlap region between 211 and 212, using glues, cements, thermopolymers, or other adhesive compositions, or they can be attached by mechanical techniques including sewing, crimping, riveting, or other fastening devices known in the art. In such a device, the body of the comb 198 can be more rigid than the sample collection tips, and can be less absorbent, providing the same impermeability to solvents used in sample analysis as is provided by an applied wax barrier as described above.

Pre-Positioned Reagents

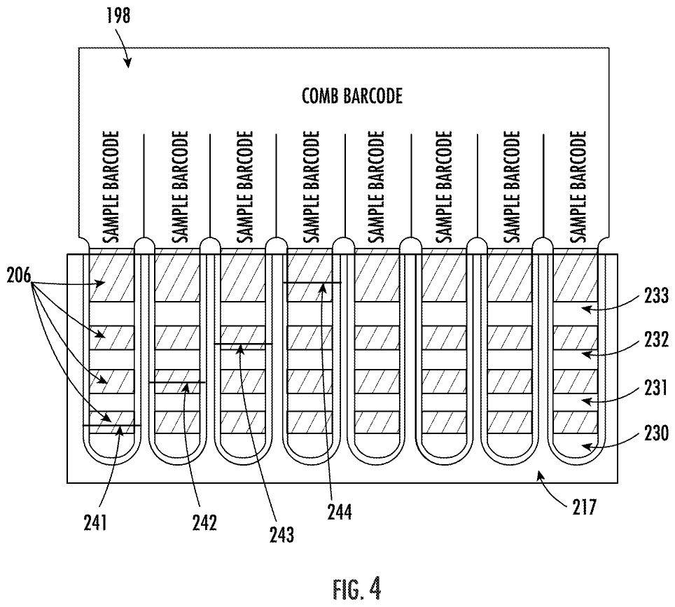

In a further embodiment shown in FIG. 4, reagents can be loaded and dried on regions of the device for later use in an analytical workflow, making use of a series of liquid, e.g., blood, impermeable barriers or barrier zones 206. In one example, the sample region (sample application zone) 230 can be pre-loaded with dried reagents that dissolve when sample is applied. For example, denaturants or detergents can be thus applied so that the sample, e.g., a blood sample, is immediately exposed to the reagents and dries after mixture with them. Examples of such reagents include CHAPS detergent, deoxycholate, urea and the like. Similarly, reagent compositions intended to stabilize sample components may be preloaded in this region, including compositions designed to stabilize RNA or RNA (for example compositions developed by GenTegra for nucleic acid stabilization), metabolites, drugs, proteins, or any potentially labile component of the sample.

In a further embodiment, the tines of the device, or zones within the tines, are coated with a composition including a nuclease enzyme capable of digesting DNA or RNA present in infectious organisms including viruses. In this case the object of the reagent's presence is to destroy a specific type of sample component rather than stabilize it. The action of such a nuclease on samples collected using the device can render the samples non-infectious by destroying any infectious genomes present, thus improving the safety of workers involved in collecting, transporting, analyzing or disposing of the samples. In this case, the use of the sample for subsequent analysis of the sample door's DNA or RNA would be impaired.

In embodiments in which reagents pre-positioned on the tines of the device preferably react homogeneously with the entire applied sample, it is beneficial to prevent the immediate dissolution of the reagent in applied sample in order to prevent the flow of sample from the application point up the tine from transporting some or all of the reagent at the front of the flowing sample zone, leading to depletion of the reagent in the part of the sample loaded last as compared to the first part of sample loaded on a tine, a process similar to common thin layer chromatography. This premature dissolution of reagent in inflowing sample can be decreased or prevented by overcoating or combining the reagent applied to the tine with a slowly soluble material such as a sugar. In this case, the sample can be loaded onto the tine in a few seconds, while the sugar-coated reagent dissolves in the sample more slowly, for example in 30 seconds to several minutes or longer. A wide range of controlled dissolution coatings and excipients are known in the art for use in slow-release drug tablets and similar applications.

Multiple reagent zones can be provided in the invention as shown in FIG. 4. In this embodiment, each tine of the device has four liquid impermeable barriers or barrier zones 206 separating liquid permeable zones 230 (where sample is applied), 231, 232 and 233 in which successive reagents are pre-loaded during device manufacturing. Such pre-loading can be accomplished by placement of a reagent solution in the zone followed by a drying step to result in dried reagent in the substrate (e.g., the paper). In one embodiment zone 230 is pre-loaded with sample denaturants (e.g., CHAPS detergent, deoxycholate, urea, or any of a variety of protein denaturants; zone 231 is pre-loaded with internal standards (e.g., stable isotope labeled peptides in the case where the sample is to be analyzed by peptide mass spectrometry) and disulfide reducing agents (e.g., TCEP); zone 232 is pre-loaded with trypsin sufficient to digest the sample proteins; and zone 233 is pre-loaded with a trypsin inhibitor (e.g., soybean trypsin inhibitor or chemical trypsin inhibitors) and antibodies designed to bind and thereafter enrich specific tryptic peptides to be measured by mass spectrometry. During manufacturing, impermeable zones 206 are printed or otherwise applied to the substrate material (e.g., paper) 201; appropriate reagents are applied to zones 231, 232, 233 in solution and subsequently dried in place. The device is then loaded by application of sample to zone 230 causing dissolution of the included denaturants which are then present in the sample as it dries in zone 230, stabilizing the sample for transport or storage prior to analysis. The analytical workflow then comprises a series of steps in which all or a portion of the tines of the sample collection device (also referred to herein as the sampling device) are inserted into wells, e.g., inserted in a 96-well plate 217, in which liquid is contacted to the respective zones in order to dissolve at different times reagents contained in the respective zones, for example, as described below. An aqueous solution (e.g., water or water containing Tris buffer, pH 7.6) is present up to level 241, after which the sample and other contents of zone 230 are dissolved in the liquid for a period of time (e.g., 1-15 minutes). Thereafter, additional liquid is added to each well up to level 242, thus dissolving the reagents in zone 231, which cause reduction of protein disulfides and add internal standards to the sample during an incubation period (e.g., 1-15 minutes). Thereafter, additional liquid is added to each well up to level 243, thus dissolving the reagents in zone 232, which causes digestion of the sample proteins to peptides by trypsin action during an incubation period (e.g., 1-120 minutes). Finally, additional liquid is added to each well up to level 244, thus dissolving the reagents in zone 233, which stops the trypsin digestion and allows the antibodies to bind the target peptides. The final result is a tryptic digest of sample proteins that includes internal standards and target peptide specific antibodies. An advantage of this embodiment (here representing the reagent addition steps of a SISCAPA workflow) is that the process only requires addition to the plate of a series of measured volumes of a single liquid (which can be water), together with controlled agitation for mixing of the liquid, sample and reagents, to effect a series of defined chemical and enzymatic steps. Agitation to mix the liquid can be carried out for example on a conventional plate shaker at a setting optimized so as to mix the liquid but not raise the meniscus into contact with the next-highest reagent layer. The resulting processed sample can be further processed to separate the antibody-bound target peptides and internal standards from the rest of the digest components (e.g., using protein G coated magnetic beads, or by recovery on a protein G column) followed by introduction into an LC-MS system for relative quantitation of target and internal standard peptides. A range of modified embodiments are envisioned in which fewer, or different reagent steps are included. For example, the device could be structured so as to carry out denaturation, disulfide reduction and tryptic digestion.

In a further preferred embodiment one or more of the tines of a sample collection comb device of the invention can be configured as a lateral flow device to measure one or more specific molecules and report a result rapidly, e.g., within 1 to 15 minutes. Such a rapid result can be used to guide collection or non-collection of subsequent samples based on a preliminary result from the lateral flow assay. Lateral flow immunoassay devices are well known in the art and typically make use of flow of a sample along the length of a linear strip of porous material, flowing across zones of reagents to carry out a series of reactions. Typically, one or more such zones are configured to report the rest result using development of color. Those skilled in the art will recognize that the zones placed along the tines of the device in FIG. 4 can be adapted to provide the series of zones (e.g., conjugate zone and test lines) used to implement a lateral flow immunoassay. The use of a sample collected by the device of the invention to carry out such a lateral flow test does not generally impact the utility of the collected sample for subsequent analysis, for example by SISCAPA. For example, the tines of the device can be configured as lateral flow assays for C-reactive protein to provide an estimate of this protein biomarker of inflammation and infection, while simultaneously serving to collect blood samples for later analysis in a laboratory. This device thus provides a point of care indication of infection while preserving a sample that can be further interrogated in a laboratory to determine the identity of an infectious agent, the amounts of other inflammation biomarkers, or the amounts of small molecules such as antibiotics. In a device in which all tines are lateral flow tests, tests on successive samples can be compared to provide a rapid indication of increase or decrease in a biomarker. In a device in which only one or a few of the tines of a page, or alternatively one or a few of the pages in a sample collection booklet, are configured as lateral flow tests, results of the test strips can be used to determine whether or when a dried blood sample should be collected on a different tine, or on a different page of the collection booklet.

In one embodiment, blood samples are collected on the tines of a device, and the tines are made of materials capable of separating plasma from blood cells (i.e. a plasma separator membrane), and the plasma emerging from the separation flows through materials with reagent zones. Such a configuration is typical in most lateral flow devices using blood as the input sample. Such a configuration also provides separate plasma and cellular samples for subsequent analysis in a laboratory, where zones of the tines comprising plasma or cellular (mainly red blood cell) components can be excised separately for analysis. Separation of plasma from red blood cells is often required in order to allow use of a colorimetric readout of the lateral flow test, for example by development of a band of color as in commercial pregnancy tests.

A multipart tine may be provided using a composite fabrication approach referred to above; i.e., by attaching a region of plasma separator material to a tine made of one or more materials suitable for flow of plasma and/or placement of reagents as used in conventional lateral flow tests.

In a further embodiment, detection of the result in a lateral flow test makes use of a visualization method that is compatible with the presence of red cells, allowing the lateral flow test to be carried out on whole blood without plasma separation. Such detection methods can include the action of enzymes or other reagents at the site of a test line that oxidize hemoglobin present in whole blood to change it from its initial red color to brown (methemoglobin, or hemichrome).

Analytical Standards

In another embodiment, the sample collection device is preloaded with standard samples in one or more positions. In a preferred embodiment, the sample zone one tine (e.g., tine H) is preloaded with a standard blood sample during manufacture and dried to yield an internal standard blood sample in each comb. After the device is used to collect 7 additional samples in the field (e.g., one week of daily samples), the comb is analyzed, and the results from the standard sample used to establish QC parameters for the samples and the processing, or to normalize the results to account for variations in sample state occurring during collection, transport, storage or analysis. In one embodiment, the standard blood sample is a pool of human blood (or serum or plasma) collected from healthy donors, applied to the sample collection device in precisely measured 15 μL aliquots and allowed to dry. Separately, the levels of selected analytes can be determined the standard sample by established independent methods, and these levels used as a single-point calibrator as described in the literature to calibrate analyte measurements in user samples.

Interface with Analytical Workflows

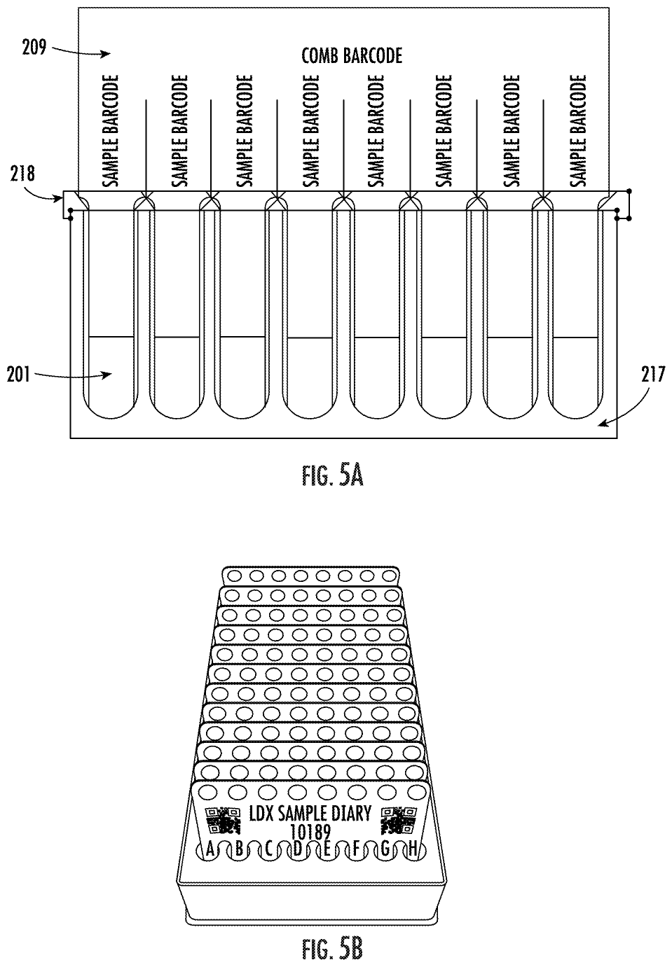

FIG. 5A shows the insertion of the comb 209 device with absorbent tines 201 into a standard 96-well plate 217. Such a plate geometry establishes a 9 mm spacing between tines, defined by an international standard configuration of 96-well plates used in laboratory robotics. A positioning plate 218 with 96 slots to accommodate the tines, can be positioned above the 96-well plate in order to allow all the sample combs to be removed (lifted) together from the plate as needed in a sample processing protocol. In total a set of 12 sample collection devices, each having 8 sampling tines, can be accommodated in a standard 96 well plate as shown in FIG. 5B. The 8 tines here labeled A-H align with the wells conventionally identified by the letters A-H in each column of the plate.

The individual tines with their dried samples can be analyzed together in 8 adjacent wells of a plate, or they can be cut apart and handled separately at the same or different times. The individual tines fit in the 96 well plate and positioning plate in the same way as the complete comb.

Book Format

Collection and storage of samples, and particularly longitudinal samples collected over time by one individual, can be facilitated by embodiments in which multiple sample collection devices can be packaged. manipulated and stored together like pages in book or booklet format, while being accessible individually for sample loading.

In its simplest form such a booklet comprises at least one sample collection comb device together with additional sheets of material that serve as protective covers or separators for the collection device(s), all bound along a common edge by a binding. The binding preferably allows the book to open and lay flat, and more preferably allows the pages to fold back on themselves to expose one sample collection device page on the opposite side of the binding from all the other pages (as in FIG. 6C). A common form of binding with these features is a ring binding or spiral binding in which the filaments of the binding pass through holes in the pages, allowing free movement of the pages around the axis of the binding while keeping the bound edges parallel. The inner surfaces of the book's covers and the separator pages between sample collection combs comprise spacers that contact the combs at points near the sample zones when the book is “closed” but do not touch sample zones themselves (e.g. spacers touching the combs within 1 to 5 mm of the samples zones), thereby ensuring that the sample zones do not contact any surface of the other pages (with the associated risk of contaminating the samples or removing some sample material), as well as providing an air gap on all sides of the sample zones to facilitate rapid drying of liquid samples applied to the sample zones (FIG. 6D). The covers and separator pages are preferably made of planar, relatively stiff, relatively inert materials such as plastics, stiff paper, thin glass, or metal. Clear covers and separator pages are preferred in order to allow visual inspection of the sample contents. A clear region is preferably provided in the front cover of the book to make visible the identifying information (including barcodes) on the first sample collection comb. The book's covers can include labels that uniquely identify the book, that identifying the subject whose samples are or will be contained in the book, or that provide information useful in using the book (including written instructions or links such as QR codes that connect the user with web-based resources useful in the course of sample collection or processing. The covers and separator pages preferably have larger dimensions than the sample collection devices so as to provide protective margins that can prevent external objects from contacting the edges of the sample collection combs. In embodiments having covers and separator pages of the same or similar basic dimensions, these pages preferably have tabs to facilitate selective manipulation of pages to open the book to a specific page or close it: for example non-overlapping tabs on adjacent separator sheets can be moved apart from one another thus opening the book to expose the sample collection comb between the separated tabbed sheets. A sample collection book of small overall dimensions, for example a size similar to a credit card and having a total thickness of 0.25 to 0.75 inches, is preferred to facilitate manipulation and compact storage.

In one such embodiment, sample collection devices, e.g., multiple planar 8-tine comb devices such as those shown in FIG. 1A, FIGS. 2A-B, or FIGS. 5A-B, can be bound together in book form to provide a sample diary in which a subject (user) can collect and preserve a succession of samples covering an extended period of time. Such a book with 4 sample pages, for example, would be sufficient to collect 4 pages×8 tines per page=32 total samples, allowing a user to collect a sample daily for a month. It will be clear to those familiar with the art that sample collection books of 1, 2, 3, 4, 5, 6, or more pages could be assembled and used in the manner described here. The sample collection device of FIG. 1A, used as a book page in FIGS. 6A-6D, in an embodiment, approximates the dimensions of a common credit card, and is therefore easy to manipulate, transport and store.

As shown in FIGS. 6A-6D, such a sample book can be provided according to the invention by binding two or more sample collection devices together to form a sample containing book, e.g., with a multi-ring spine, e.g., as a miniature version of a ring-bound notebook held together by a multi-ring binder spine such as Swingline GBC Proclick Binding Spines 402 (as shown) in which the 8-tine sample collection devices are pages. When using such a binding method, the individual sample collection device pages can be pre-punched with an array of holes through which the rings of the binder pass. In the book format shown in FIGS. 6A-6D, a series of up to 10 holes is punched to accommodate the spacing of the rings in Swingline GBC Proclick Binding Spines; however, a smaller number of rings (and associated holes) can be used, down to a minimum of 2 rings necessary to maintain the pages in a book-like configuration. An advantage of such a ring-bound format is the ability of the book pages to be folded back in such a way that a single device extends away from all the other pages (FIG. 6C), allowing improved access to the extended device page for unobstructed sample application. Alternatively, the devices can be spiral-bound, held in a 3-ring binder, or sewn in signatures like a book.

Preferably non-absorbent separator pages 403 (FIG. 6D) are bound between the sampling device pages to prevent any possibility of contact or transfer of sample material between sample pages. The separator pages 403 and covers 405 in the example shown in FIG. 6D are made of 0.020″ polycarbonate cut using a steel rule die, but they can be made a variety of rigid or semi-rigid, clear, translucent or opaque materials including paper, plastics such as polycarbonate or polypropylene, or metal. In order to prevent contact of the samples on the collection device pages with the separator pages or covers between which they are bound, separators between the collection device pages and separator pages may be used, e.g., spacer strips 404 are applied to the faces of each cover or separator page that faces a sample device. In the example shown the spacer strips 404 are made of 0.030″ polyethylene tape having an adhesive on one side that sticks it in place on the polycarbonate. By holding the samples spaced away from the covers or separator sheets on either side, an air gap of ˜0.030″ is formed above and below the sample (as shown in FIG. 6D), preventing contact with the separator pages and coves, and facilitating the drying of the samples. Each sample page is thus sandwiched between a cover or separator sheet on either side separated by an air gap. These airgaps can preferably be 0.020″ to 0.030″, or they can be as small as 0.005″ and as great as 0.5″ according to the geometry of the book and the requirements of air exchange for drying. The spacer strips are preferably positioned (FIG. 6B) so as to be near the zone of the sample device tines occupied by sample, thus minimizing the risk of a bent sample tine contacting the separator page or cover, but not close enough so that the spacer strip itself touches sample when the book is closed (including possible relative motion of the pages due to the looseness of the holes around the rings of the binder). A variety of materials and methods can be used to form the equivalent of these separator strips to generate the desired spacing. Tapes of various materials including polyethylene, vinyl, or other plastics, paper or other fibrous or non-fibrous materials can be adhered to the separator pages and covers in the desired position. Likewise the material of the separator pages and covers themselves can be formed with integral separator strips, e.g., by molding (including injection molding), thermoforming, cold forming (e.g., of polycarbonate sheet); by application of a liquid material such as a thermosetting plastic or a polymerizable composition that solidifies forming a “bead” of the required dimensions, or by embossing or debossing.

Tabs 401 can be formed on, or applied to, the covers 405 and separator pages 403 to facilitate access to specific sample devices (pages) inside the book. The covers of the book can incorporate a closure to keep the book closed when stored.

Separator pages of the sample book (i.e., not the sample collection combs) can be made of water-proof materials capable of being cleaned and recycled, thereby reducing the cost of the sample book.

Book Including Desiccant

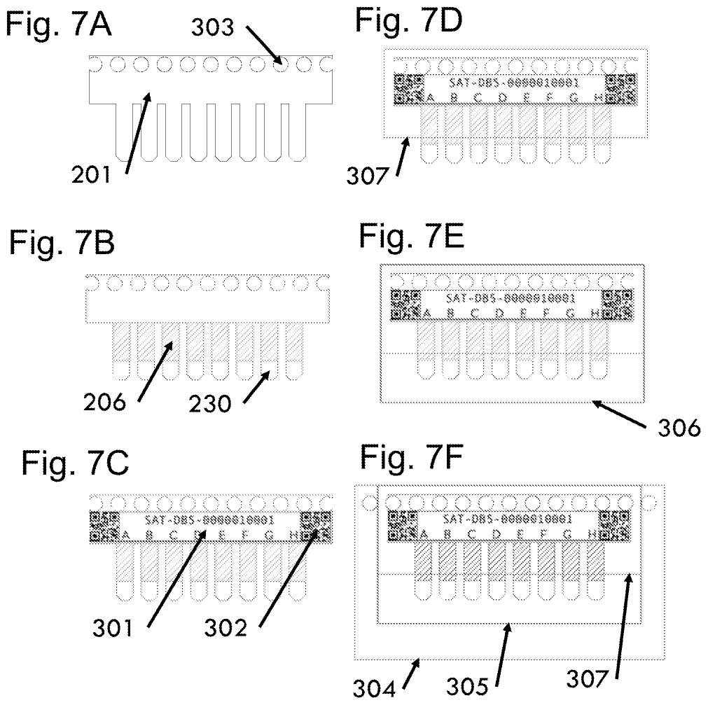

The assembly of a related alternative book format embodiment is shown in FIGS. 7A-7F. FIG. 7A shows a planar tine 201 made of sample-absorbing material. In an example, this material is Whatman 903 paper and the component 201 is cut from a commercially available 903 Protein Saver card. Holes 303 punched in this region accommodate rings for binding the component with other layers into a book.

FIG. 7B shows the application of surface wax to regions 206 of the sampling tines to define a sample application zone region 230. Blood or other liquid samples applied to 230 will fill the paper up to the edge of regions 206 but not penetrate beyond. The area of sample regions 230 thus define an approximate sample volume, based on the area of the region and the thickness of the paper.

FIG. 7C shows an identifier, e.g. a label 301 e.g., applied to (or printed directly on) the device, and includes one or more machine-readable labels. In the case shown, 2 copies of a 2D QR code 302 encoding the card identifier (shown as “SAT-DBS-0000010001”) are included (a wide range of alternative machine-readable codes can also be used). In addition, in the example shown, the 8 sampling tines are identified by letters A-H, allowing them to be unequivocally matched with wells A-H of a row of sample wells in a conventionally labeled 96-well plate. Other labeling schemes can be used, including numbers, and each of the 8 tines can be provided with its own individual machine-readable label (e.g., a linear barcode). The device label in the example shown also includes a human-readable text label. In the example shown, the height of the label and perforated region above it has been minimized in order to facilitate access of robotic pipetting devices to 96-well plate wells in which the device is inserted (i.e., reducing the vertical height of the sampling device above the top of the 96-well plate as shown in FIGS. 5A-B).

In a further embodiment, a desiccant can be used to dry sample contained in the sample collection device which desiccant may, for example, be placed in the book. FIG. 7D shows such an embodiment with the relative size and position of a rectangular piece 307 of planar desiccant (e.g., Sorbent Systems desiccant paper grade 105 or 460) which can be placed above, below or otherwise juxtaposed with the sampling device (for example by binding into book form as successive pages). This desiccant is shaped and placed so that it cannot contact the sample-loaded region 230, but is in close proximity to it, thus enabling rapid and effective drying of the sample through transfer of water through the intervening atmosphere. Placement of desiccant in the book itself is an alternative to use of a desiccant placed in a closed container outside the book, as described in other embodiments lacking desiccant pages within the book.

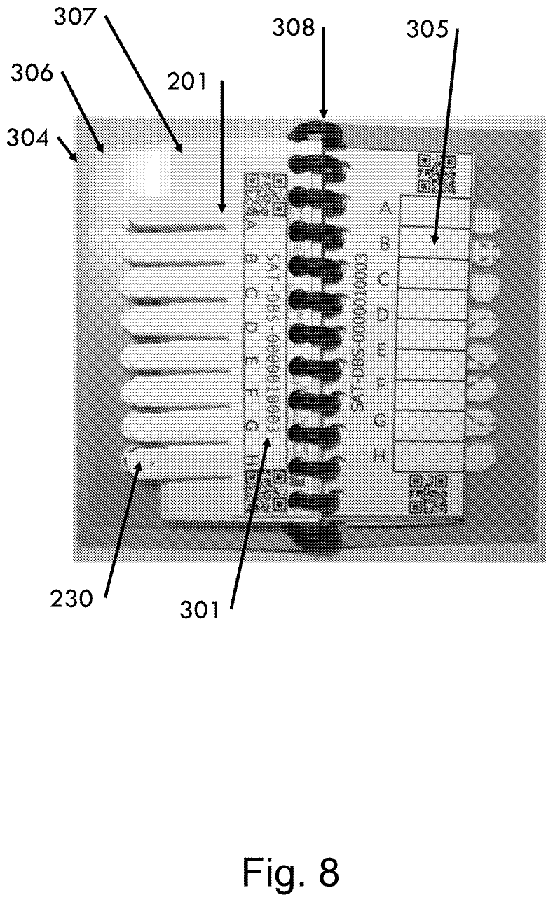

Shape 307 is also representative of a preferred shape for a sheet of material (e.g., a page of paper 305 for recordation of information as shown in FIG. 8) bound together with the sample device for recordation of information such as date of sample collection, etc. Scanning such sheets, either in the field or upon receipt of the sample book at a processing laboratory, allows facile recordation of the information in digital form. Placement of manually entered data records as pages within the book is an alternative to the use of electronic means as used in other embodiments described herein.

FIG. 7E shows the size and placement of an inert separator sheet 306 that can be included to prevent contact between adjacent sampling devices when these are stacked or bound together. Such a separator sheet is preferably made of a material to which sample does not stick and which does not contain contaminants that could adulterate the sample in the event that a sampling region 230 were to touch the separator sheet in a stacked or bound configuration.

FIG. 7F shows the relative size and position of a cover 304 designed to protect the contents of a bound book of one or more sampling devices, together with any desired additional “pages” of desiccant 307, sample information sheet 305, separator sheets 306. In a preferred embodiment, multiple sampling devices 201 are bound together by a binding to form a book. The binding is preferably one that allows pages to lay flat or to be made to project from the other pages allowing clean application of samples: examples include spiral or coil binding, or GBC ZipBind binding spines as shown in FIGS. 6A-6C.

FIG. 8 shows an example of such a book, in which 4 sets of pages (each including a sample collection device 201, a sample information sheet 305, a desiccant sheet 307, and a separator sheet 306) are bound between covers 304 by ring binding spine 308. It will be clear to those skilled in the art that many additional forms of information can be collected in such a sample collection book, and that this information can be translated into digital form through the interpretation of photographs or machine readable codes (e.g., by a smartphone or computer vision system) in a variety of locations (e.g., at the site of sample collection, in transit, or in an analytical laboratory).

Sample Loading in Book

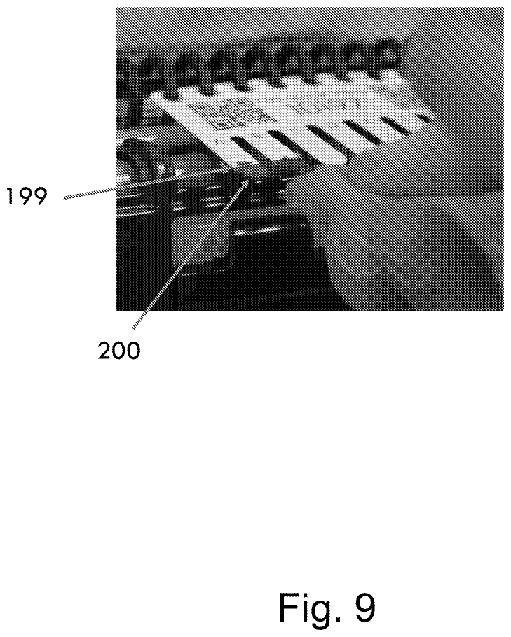

A useful feature of a ring-binder or so-called “lay-flat” type binding is the ability to expose one page opposite all the others, thus facilitating placement of samples on a specific site on one collection device (page) without touching others. FIG. 6C shows such a book rearranged so that one page hangs down separated from all the others. The book can then be laid down on a convenient object with the tines extending out nearly horizontal as shown in FIG. 9 to facilitate sample loading. This sample-loading geometry offers a significant advantage over the orientation used in collecting finger-prick blood onto conventional dried blood spot cards. Blood emerging from a lancet fingerprick can be applied to the sample application zone end 200 of a sample collection tine in full view of the user: the finger is held so that the blood droplet is visible on top of the finger, which can then be brought up to the sample application zone end, e.g., tip of one of the tines of the comb device, whereupon blood flows from the droplet into the edge of the sample application zone in a manner and speed that the user can control. Thus in the device of the invention, the application of blood takes place in full view of the user, who can regulate the amount of blood applied by touching the blood droplet to the edge of the device or withdrawing the finger, and thus apply an amount of sample that extends up to a line 199 on the device tine that represents the target sample volume to be obtained. In contrast, when loading a conventional dried blood spot card, the blood is applied to the card by dropping a droplet from the invisible underside of a finger onto a region of the card obscured by the finger, resulting in user uncertainty as to the position and amount of sample applied.

FIG. 9 shows a book format embodiment arranged so that sampling tines of one page comb project from the book, in this case to the side away from the other pages. This configuration enables easy access to each of the 8 sample tines on the page without contact with any of the other pages. In a preferred embodiment shown in FIG. 9, samples are applied to the device when the sample device tines are approximately horizontal, or with tips inclined slightly downwards from the spine, so that sample (e.g., blood) is applied at the tip of the sample device tine and wicked from the tip towards a desired fill line 199 where the blood flow is intended to stop. This line can be defined as an embossed or crushed line (as shown in the figure), as a printed line, or by the edge of a hydrophobic (e.g., wax) barrier applied to a paper sample collection device. When sample is applied in this way any tendency to overload the device (which could occur due to the force of gravity if the tip of the sampling device were inclined upwards) is minimized. The application of sample to the device in full view of the user is a significant advantage of the device used in this way.

Samples can also be introduced into the imbibing zone 200 of the sample collection device by dispensing from a user operated pipette (e.g., an Eppendorf pipette) or by placement of a viscous or solid sample rather than direct application by user contact.

Book Disassembly

Sample books made according to the invention can be easily disassembled after application of samples to facilitate their analysis. In book embodiments shown, the binding used (Swingline GBC Proclick Binding Spines) can be reversibly opened using a device provided by the manufacturer, or alternatively by hand, to allow removal of pages. Disassembly of the sample books can be automated in a variety of ways including insertion of guide pins though the binding holes to retain the pages in position while the binding is removed, followed by sequential removal of pages, for example using a vacuum grasper to individual pages in order and place them in appropriate locations for subsequent processing. By opening the binding and removing the pages, sample device pages can be accessioned into a laboratory for analysis (identified by the page bar or QR codes), while the covers and separator pages can be reused if desired by assembling them with fresh unfilled sample collection pages, thus helping reduce the cost of sample collection books by recycling most of their components. As described, the sample collection device pages can be directly inserted into columns (or rows) of a standard 96-well plate (FIG. 5B), causing the sample containing regions 230 to be placed at or near the bottoms of the wells, accessible to liquid that has been placed in the wells either before or after insertion of the sample collection device. A variety of workflows can be carried out using the samples collected on devices according to the invention, and the devices can be withdrawn (removed) from the wells at any of a number of stages during a workflow. This withdrawal can be accomplished either by grasping each sampling device and removing it (thus removing one set of sample tines), or withdrawing multiple devices at once by raising a thin plate 218 in FIG. 5A with 96 holes placed on top of the 96-well plate, and through which the sampling device tines project into wells in the 96-well plate. The sampling devices can also be stored, either bound in books or separated, for later analysis.

Sample Storage

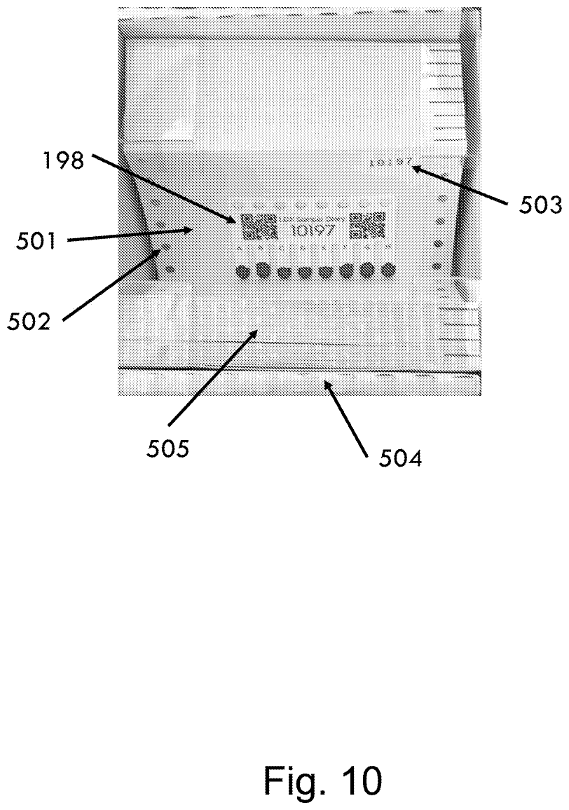

It is useful to be able to store devices according to the invention, including collected dry samples, for later analysis, or as an archive for later study. It is desirable to store the sample devices dry, to protect them from contamination, to organize samples for rapid access of any specific sample in a compact catalog format. In a preferred embodiment, devices according to the invention are stored in a compact file similar to a library card catalog, made from fan-folded cardstock. FIG. 10 shows an example of such a system, in which a continuous strip of 250 fan-fold cards (505) is folded into an accordion that provides 125 slots, each capable of holding a sample card 198 in isolation. In this example the fan-fold material is 4″×3″ Compulabel® 210104 Continuous White Name Badge Cards—Pinfeed. The material has pinfeed sprocket holes 502 on both sides suitable for feeding the material through a dot-matrix printer (e.g., when used for printing name badges). In a preferred embodiment, every second page of the fan-fold is printed with a consecutive number 503 (in FIG. 10 this is 10197) that is the same as the number readable by a human or by a computer on the sample card 198, thus providing a pre-designated location for that card that is easily found by searching sequentially through the fan-fold stack. Since the fan-fold pages are connected at the bottom of each position, the sample comb cannot fall out the bottom, and is surrounded by a wide margin of clean cardstock. In this compact format, a set of 125 sample cards (1,000 total samples) can be stored in a 5″×5.5″×3.5″ box (FIG. 10). The desiccated storage space requirements for large sample collections, either refrigerated or at room temperature, are thus substantially reduced compared to conventional samples.

In a further preferred embodiment, samples can be maintained for long periods at low humidity, and in an oxygen-free atmosphere by purging the storage system with a gas such as nitrogen or argon, thus displacing any air (including oxygen) with an oxygen-free dry gas atmosphere.

In a further preferred embodiment, the fan-fold paper can be supported by rods or cables running through the pinfeed holes and allowing easy sliding of cards in the course of searching for specific numbered slots.

Subdivision of Samples

In some circumstances it can be desirable to remove a portion of collected samples for analysis, while retaining the remaining sample together with the associated identifying labels. For example, a portion of each sample collected on a sample comb can be removed by cutting off the tip of each tine and depositing the removed material into the wells of a 96-well plate for processing, while retaining the remaining sample on the comb (e.g., stored in the fan-fold storage system described above). It is convenient to slice the tips off the 8 tines at one time, for example by using a pair of scissors aligned perpendicular to the tines, and held above the desired column of wells of a 96-well plate. More preferably a parallel cutting device capable of cutting a uniform length off the end of all 96 tines in twelve 8-tine sample devices can be used, this device being positioned over a receiver plate into which the excised samples fall. Such a parallel cutter can use any of a variety of cutting mechanisms applicable to paper that are known in the art (scissors, blades, shears, etc.).

Alternative cutting means can be used that produce a circular punch, for example a 5/16″ punch as used in notebook hole punches. Such punches can be used individually to cut a subsample from one sample at a time, or as a group, e.g., of 8 punches spaced 9 mm apart so as to excise a similar region of the 8 tines of a sample comb as described above. Various methods are available to clean cutters between uses to as to minimize any cross-contamination between samples.

Alternatively, in a preferred embodiment a series of sample combs (e.g., 12 corresponding to 96 samples destined to be placed in a 96-well plate for analysis) can be arranged side by side with the sample tips projecting forwards. A computer-controlled cutting device may be used to clip a portion of each tine into a receiving vessel (e.g., a well of a 96-well plate) below. Computer-controlled motion systems can be used to move the cutter from tine to tine along the series of combs, while an X-Y table beneath the cutter aligns the desired well beneath the cutter to receive a sample.

In a preferred implementation of such a system, a camera images each tine from which a sample is to be removed according to a specified worklist, and a computer program assesses the amount of sample dried thereon, typically by assessing the area of the sample zone.

The computer then causes the cutter to be positioned so as to excise an area of the dried sample responding to the amount of sample required for the assay to be conducted. Photographs can be taken of each device before and after removal of subsamples, and image analysis of these photographs performed to measure and/or verify the amount of sample removed, and the amount that remains as an inventory for possible future use.

Sample Collection Kit & Housings

The sample collection devices of the invention, separately or in book form, can be kept in a closed container with a desiccant to ensure rapid drying and dry storage. Such a container naturally forms a convenient kit in which the sample collection devices, desiccant and other components can be organized, delivered, and housed when not in use.

In its simplest form, a kit comprises a reversibly openable airtight box with sufficient internal space to accommodate a sample collection book and a quantity of a desiccant sufficient to extract a large fraction of the water present in samples applied to the book, thereby drying them. The desiccant is preferably capable of reducing the relative humidity (RH) inside the box to less than 10%, less than 5% or most preferably less than 2% or less than 1%. The box preferably has a sealing gasket that provides an air-tight seal when the box is held closed by a reversible clamping system. The kit optionally also comprises a lancet capable of piercing human skin to generate a droplet of blood. The kit optionally also comprises an RH indicator visible from outside the box. The kit optionally also comprises an electronic logging device capable of sensing and recording RH and/or temperature and reporting this information to an external digital communications or storage system. The kit optionally also comprises an electronic camera capable of photographing a freshly collected sample. The box can optionally serve as a shipping container that can be delivered to a subject for collection of samples and returned to a sample repository or analytical laboratory.

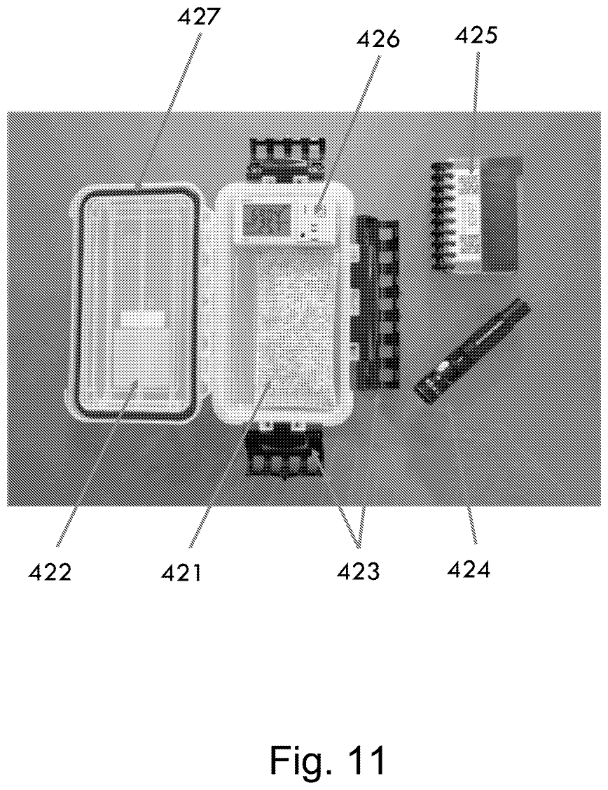

In a preferred kit embodiment shown in FIG. 11, the sample collection kit comprises an airtight box 422 capable of holding one or more sample collection devices or books 425 and a quantity of desiccant, preferably molecular sieve in a mesh bag 421. The box comprises as two-part hinged shell with a gasket 427 and clamps 423 providing an airtight seal. The box preferably has an internal volume close to the volume required to hold the kit contents, since excess volume allows a greater volume of ambient (humid) air to be introduced each time the box is opened. At the same time, the box and its contents are preferably shaped so that water escaping from samples in the sample collection device or book can migrate rapidly to the desiccant by diffusion in the air (i.e., that there be minimal physical barriers between fresh samples drying in the collection device and the desiccant. The exterior surface of an airtight box can be sterilized if necessary (e.g., by washing, dipping in dilute bleach, or exposure to ozone) without affecting the samples contained inside, or exposing staff who handle the closed box to any pathogens that could have been present when the box was opened while collecting samples in the presence of an infection. In FIG. 11, the box shown is a commercially available Plano 3440-10 box typically used for protecting cellphones in damp environments. Such a box is physically robust and can be made to function as a shipping container (e.g., for US mail, Federal Express, UPS or Amazon transport) without the need for added externally packaging or labeling.

In another preferred embodiment, the airtight box that serves as a package for the sample collection kit is made opaque so as to prevent or diminish the exposure of collected samples to light. The opacity can be achieved by application of an opaque paint or other coating to an otherwise transparent or translucent box, by fabricating the box of an opaque material, or a variety of other means known in the Art for container manufacturing. If desired a portion of the box housing the sample collection device(s) can be made opaque, while other sections of the box remain clear so as to allow visual observation of objects or devices, such as a data collection device, inside the box.

While, in principle, the use of an airtight box allows complete control of the humidity of the environment inside the box, there are nevertheless practical constraints on the relative humidity (RH) values that can be reproducibly provided in such a small volume. The use of powered humidifier/dehumidifier mechanisms is impractical given the space, power, drain and input water requirements of such systems. Precise equilibrium RH values in the range of 3%-30% can be achieved through use of slurries of specific salts, but these are likewise difficult to provide in a small, physically robust package. Hence from a practical perspective, the most robust approach for providing a truly reproducible RH environment is to remove essentially all water from the air of the enclosed box—i.e., to achieve ˜0% RH. Zeolite “molecular sieve” desiccants can provide this capability because of their very high affinity for water and large adsorptive capacity.

Biological samples dried at low relative humidity are stabilized and can survive storage and transportation unrefrigerated for weeks or months. Protein structure is expected to be preserved at 0% RH for extended periods at ambient temperature, as indicated by studies on the stability of recombinant therapeutic proteins (Breen, 2001), the observed long-term viability of vegetable seeds at room temperature and above when RH is reduced below ambient (Ellis, 1989), and because of water's well understood role in many degradative chemical processes. In most biological samples, achieving very low RH involves removal of a large amount of water: blood typically contains 85% to 90% water.

In the airtight box kit described above, the desiccant is preferably an efficient desiccant such as molecular sieve 4 A Blue Indicating Molecular Sieve Desiccant (Delta Absorbents) or tabletized molecular sieve 4 A (Sorbent Systems) or molecular sieve 3 A. The quantity of molecular sieve should be sufficient to remove a large fraction of the water from the samples to be collected. In the case of the preferred embodiment shown in FIG. 11, 50 g of 4 A molecular sieve is used, contained in a silicone mesh bag 421 so as to be able to capture water vapor from the surrounding air in the box. Such silicone mesh material is commercially available in the form of non-stick dehydrator sheets used for drying fruit, and the material can be formed into bags by sewing and/or gluing with silicone adhesives. Given the ability of molecular sieve to adsorb water equal to 20% of its weight, this amount of molecular sieve can theoretically absorb 10 g (=10 mL) of water, which is >20× the amount of water in 32 samples of ˜15 uL (roughly 0.250 mL water). This excess provides added desiccant capacity to remove any water introduced into the box when the lid is opened to remove or replace the sample book or lancet inside. An advantage of using a silicone mesh bag is the ability to recycle the desiccant, either by heating in an oven to ˜250 F, or heating in a microwave oven, to expel the adsorbed water from the desiccant and thus allowing desiccant reuse. Alternatively, a cloth mesh bag, or a Tyvek bag can be used if the desiccant does not need to be recycled (i.e., is treated as a consumable). In a further preferred embodiment, the molecular sieve desiccant is formed into a solid shape, e.g., a rectangular plate, which can be fastened to the inside of the box. Such a solid form can be easier to manufacture in volume than a mesh bag of molecular sieve beads.

Many desiccants are available in a form that includes a humidity indicator (frequently a blue color that turns to pink or beige when the desiccant has absorbed its useful capacity of water)—such an indicating desiccant is preferred since it allows a user to know that the desiccant is active (i.e., blue) when the sample is loaded onto the absorber, as well as showing that the desiccant retains some additional capacity after the sample has been dried (and hence can maintain a low humidity in the sample vessel during storage and transport). Using a sufficient quantity of desiccant, the sample can be rapidly dried, stabilizing it for later analysis.

A second advantageous characteristic of molecular sieve desiccants, particularly in granular (bead) form and provided in highly porous container such as the very open silicone mesh bag 421 shown in FIG. 11, is the speed with which water is removed from the air inside the box and the corresponding rapid drying rate of samples collected on the devices described here. Measurements collected inside the box by a RH data logger indicate that the configuration shown results in a rapid decline in RH from ambient (i.e., the RH of the air introduced into the box when opened to place the sample book inside) to <1% RH in 10-30 minutes. This rapid drying decreases the time during which degradative processes can occur in the samples before they reach a state of dryness.

In addition to a quantity of desiccant, an oxygen absorber (powdered iron or preferably a polymeric oxygen absorber that does not release water) may be included in or with the desiccant vessel in order to eliminate free oxygen in the vessel after sample collection, thereby reducing the potential for chemical oxidation of sample molecules during storage. Minimizing post-sample acquisition oxidation of protein methionine residues for example, can help preserve potentially relevant biomarkers related to in vivo methionine oxidation. The oxygen concentration inside the container can be measured and reported by a variety of means including colored reporter compounds, electronic oxygen meters, and the like.

In a further preferred embodiment, the kit contains lancets capable of puncturing skin to generate small samples of capillary blood. In the kit embodiment shown in FIG. 11, a Roche AccuChek FastClix lancet device is included, which provides for user selection of penetration depth. This lancet device contains a canister of 6 lancets internally and is capable of advancing from one to another through a simple click. Additional canisters (e.g., 5 additional canisters to provide 36 total lancets covering 32 sample collections) can be included in the box as well. This lancet is FDA-cleared, involves minimal pain and reproducibly generates sufficient blood to fill a tine of the device shown in FIGS. 6A-D.

In a further preferred embodiment, the kit also contains a small battery-powered digital data logger 426 capable of measuring and recording relative humidity (RH) and temperature, and delivering this log in electronic form via Bluetooth connection to devices such as cellphones. The device shown is an Onset MX1101 RH/Temp logger capable of storing measured RH and Temp at regular intervals (e.g., every 5 minutes) for extended periods (months). A profile of RH values measured by this device during a month's use of a kit described in this embodiment is shown in FIG. 12. A series of peaks 430, generally with maximum RH>10%, indicate times when the box was opened and external (i.e., ambient) air was admitted, causing a brief increase in RH until the box was closed. These events provide a direct record of the precise times and dates of box opening and closings, which in turn indicate when the sample collection book was removed from the box and then put back. In the kit configuration shown, the RH rapidly declines from each peak to below 5% RH, and then declines to achieve a steady state value very near 0% RH. The timecourse of the final RH decline (e.g. the trace indicated by label 431) provides an indication of the timecourse of extraction of water from the freshly introduced sample. Such traces are useful for establishing the precise time each sample was acquired (box was opened and closed), whether the kit was opened on a given day (i.e. whether a sample was collected), whether the sample dried at the rate expected. Together with the temperature, which is similarly recorded, the RH trace records the storage conditions for each sample since the time of its collection, providing a valuable source of data for the study of sample quality and the determination of quality metrics and controls to ensure sample quality for analysis.

Sample Recording by Mobile Devices

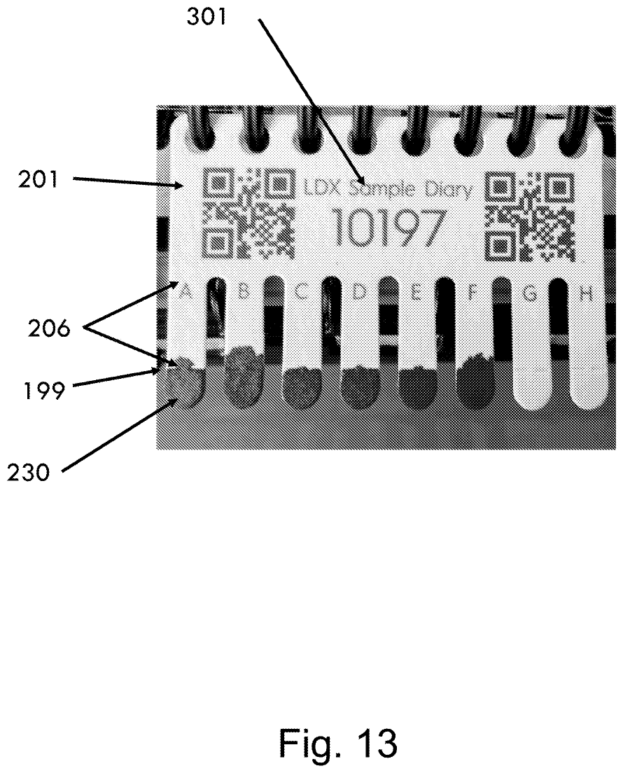

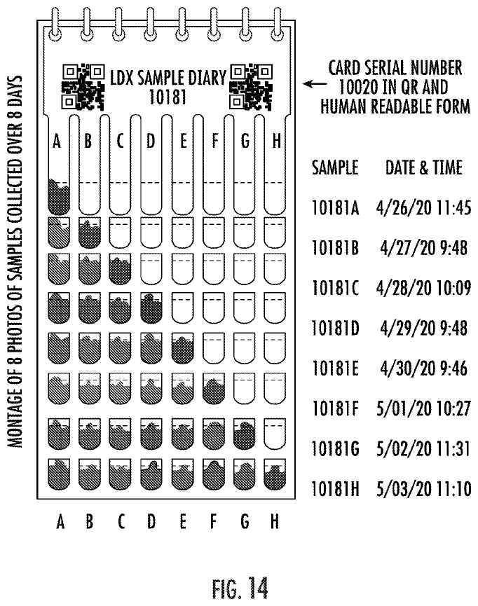

In a further preferred embodiment, a digital camera such as that found in a cellphone is used to take a photograph of each sample immediately after collection (i.e., before the sample collection device is replaced in the kit box). FIG. 13 shows such a cellphone photo, which clearly records the sample collection device label and QR code, and the sample content of each sample device tine (facilitated in the case of blood samples by the red color; in this case 6 samples have been loaded at one time on tines A-F). FIG. 14 shows a montage of 8 such photographs taken over 8 days, i.e., as each of the 8 samples collected on the 8 collection device tines was acquired. The time and date of collection was extracted from the cellphone image in each case and is listed for each sample. In these images it is clear that each day's freshly collected sample is red, and any previously collected sample is brown, a finding consistent with the known conversion of oxyhemoglobin in fresh blood to methemoglobin and ultimately hemichrome in dried blood during 24 hours. This color information provides a further indication of the age of each sample at the time the photograph was taken; a sample that is red in a photograph was collected recently (less than 12-24 hr. previously). While it is likely that the illumination conditions may be variable from picture to picture due to differences in time of day, location of the sample collection device, weather, season, etc., the presence of areas of unoccupied white paper substrate provide a means of adjusting the white balance of each photo to bring all onto a consistent scale of color, thus allowing quantitative analysis of sample color information.

Photographs of collected samples also provide a means of estimating the amount (i.e., approximate volume) of each sample by measurement of the area of the blood color. Numerous computer algorithms exist to measure the area of connected regions of a photo defined by a consistent feature such as color (red used here for whole blood). This information can be used in the laboratory during sample processing to determine whether adequate sample is available, whether a subsection of the sample (e.g., the tip of the tine) can be removed and still leave sufficient sample for further analysis, etc.

In a further embodiment, an app on the smart digital device is capable of collecting additional data via various interfaces such as Bluetooth, Wi-Fi, etc. For example, a cellphone app can interrogate a humidity sensor or logger inside the kit container periodically, when samples are collected and photographed, or at other intervals, and adding this data to a cloud-based data repository where other sample-related data (e.g., photographs) can be accumulated for use in combination with a sample's analytical results.

Similarly, the mobile device app can additionally record the subject's action or report of taking prescribed medication, either in the context of a clinical trial or as a patient. Clinical trial subjects collecting samples using the invention can optionally conduct additional interactions with a mobile device. In a preferred embodiment, such trial subjects are provided with treatment materials including drugs in a format allowing self-administration synchronized with sample collection. For example, a drug to be administered once per day can be taken at the same time as a sample is collected, prompted by a reminder delivered via a mobile device. In this embodiment the drug can be packaged and delivered together with the sample collection kit. Adherence to both administration and sample collection protocols is reinforced by the connection between the two.

Many advantages flow from the direct association of a smart digital device such as a cellphone, iPad, etc. (mobile device) with the act of collecting a sample (e.g., a capillary blood sample). The association of the user (in general the owner or authorized user of the phone) is established using security features native to current generation phones (fingerprint ID, face recognition, etc.). The time, date, and GPS coordinates at the moment of collection are easily recorded, and in many cases appear in the headers of digital photos taken by the phone by default, thus providing this information along with any pictures taken of sample collection devices as described above. Voice comments provided by the user, including useful health-related contextual information, can be attached to photos, recorded as sound files, or converted directly to text by automated means accessible from the phone. Contextual data such as heart rate and levels of exercise can be collected directly by the user's smartphone or an attached device such as an Apple Watch or Fitbit, and additional data such as weight, blood pressure, blood glucose, etc. can be measured externally and collected by the smartphone (e.g., using the Apple Healthkit) as context in the interpretation and use of biomarker data generated from blood samples collected according to the invention. A variety of other sensors can be incorporated through future integration with such mobile platforms. The ability of current cellphones to provide digital data transmission to cloud-based software and data storage facilities, particularly facilities compliant with HIPAA privacy rules, enables the systematic collection of photographs, voice, text, and health parameter data associated with blood samples collected using the present invention.

Another preferred embodiment of the collection kit comprises a machine-readable QR code on the kit box or sample collection booklet that can be interpreted by a mobile device to yield a link to a cloud-based resource for collection of digital sample-related data. Many smartphone cameras automatically recognize such QR codes and open a specified webpage, facilitating user access. In one preferred embodiment, scanning a QR code using a cellphone provides access to a web-based form or cellphone app into which a user enters information about current health status, time and location of each sample as it is collected, as well as a photograph of the applied sample. Such a resource can be provide using facilities such as Google Forms accessed through a smartphone browser, or more sophisticated and secure HIPAA-compliant software systems (e.g., Crucial Data Solutions' Trialkit or REDCap Cloud platforms). A smartphone can also acquire an image of the sample donor as part of a permitted sample collection protocol and add this photograph to the digital record as verification of the donor's identity. Smartphones also facilitate protocols in which the sample donor is reminded (e.g., by a calendar app) to collect a sample at a certain time or on a certain date.

Sample books according to the invention can also incorporate a remotely readable identification chip or other device allowing electronic identification of the book or individual sample collection device sheets (for example an RFID chip).

A cellphone, particularly a smartphone, can also be used to provide the user with pictures, video and audio help with the sample collection process itself. Examples include the action of preparing and lancing a fingertip to provide blood, the operation of the collection device and the subsequent storage and shipping to an analytical laboratory. This help can be pre-generated or can include live contact with a human assistant.

A machine-readable code such as a QR code can also be used to connect the user directly to a website or other digital portal through which the user can obtain or interact with his or her personal data and analytical results. For example, analytical data resulting from analysis of samples using the device, including longitudinal data collected as a series of samples over time, can be delivered to a subject, healthcare providers or others via mobile devices including cellphones, iPads, and the like. In a preferred embodiment, prior results from a subject's serial samples and/or other contextual data are interpreted by computer algorithms (or by human consultants) and the conclusions of this interpretation are used to determine the timing of collection of subsequent samples. For example, if a potentially significant change in observed in the level of a biomarker in the most recent sample, or if a subject's contextual data shows a major change in temperature, mobility or resting heart rate, the subject could be informed as a result of the data interpretation that it is advisable to alter the frequency or timing of future sample collection, e.g., to collect another sample soon in order to confirm the existence and/or magnitude of the suspected change, or to perform tests of additional biomarkers. Establishment of a closed loop of communication between subject and analytical result provider offers a major improvement in the user experience and in the performance of tests carried out on the samples.

It will be evident to one skilled in the art that the capabilities described above as useful in a sample collection kit (e.g., an airtight box, a sample collection device, a desiccant, a lancet, a data logger, and an associated mobile device such as a cellphone) can be simplified, miniaturized, and integrated in a purpose-built device that is less costly to produce, smaller, lighter, easier to ship and potentially easier to use. In one such embodiment, the kit comprises a specially designed plastic airtight box containing a solid block of desiccant, a miniaturized electronic package comprising sensors to measure RH, temperature, and GPS position, take photographs of fresh samples, connect to a digital data network (e.g., via WIFI, Bluetooth or cellphone connection) and acquire spoken audio (e.g., voice commands and contextual comments and data). Such an embodiment can be substantially smaller than the box shown in FIG. 11. In a further embodiment, the foregoing box can form the covers of a sample collection device: i.e., the covers of the book can also be shaped in such a way as to provide, when closed, an airtight container for the samples. In this embodiment, opening the box exposes the sample collection device page(s), one of which can be presented for application of sample, after which the box takes a photo of the fresh sample and the box is closed while the sample dries.

Alternative Housings

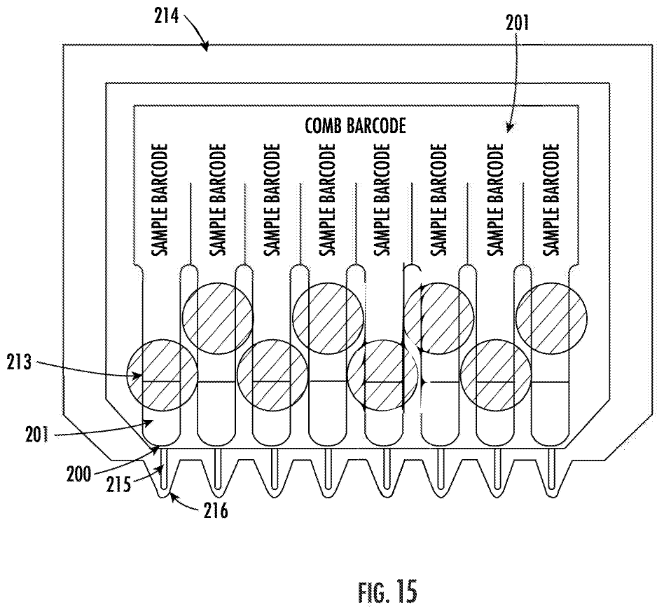

Some applications may not require multiple sample collection comb pages, and may thus not require a reconfigurable format like the pages of the booklet shown in FIGS. 6A-6D. For applications in which a single comb device can be used (e.g., for collection of 8 or fewer samples), alternative device housings can be provided that do not require an openable box. In one preferred embodiment, the sample collection comb device is contained in a housing as shown in FIG. 15. Here comb sample collection device 201 is placed inside a flat package 214 formed of two sheets of impermeable, moldable material such as thermoplastic sheet that has been formed to provide a cavity for the comb, positions for desiccant (in this case shown as eight standard molecular sieve tablets 213), and capillary channels 215 that contact the tip 200 of the sample collecting tines 201 on one end and come near to the outside sealed edge of the package 216 at the other end. The capillary channels are arranged so that by snipping off the end of the projection 216, the end of the capillary is exposed. Blood applied to this end is pulled into the capillary by capillary forces and delivered to the respective adjacent tine absorbent zone 200 which then imbibes blood. In one embodiment, a barrier line 206 (as in FIGS. 2A and 2B) stops blood inflow, establishing an approximate sample volume. After sample is loaded, the end of the opened capillary can be closed by application of a cap, or left to be sealed by drying of the remaining blood in the capillary to form a solid plug. The package 214 is so designed as to maintain the sample comb near to but not touching the desiccant 213 that is placed above or below and separated from the sample absorber by a narrow air gap, thus allowing rapid drying of samples.

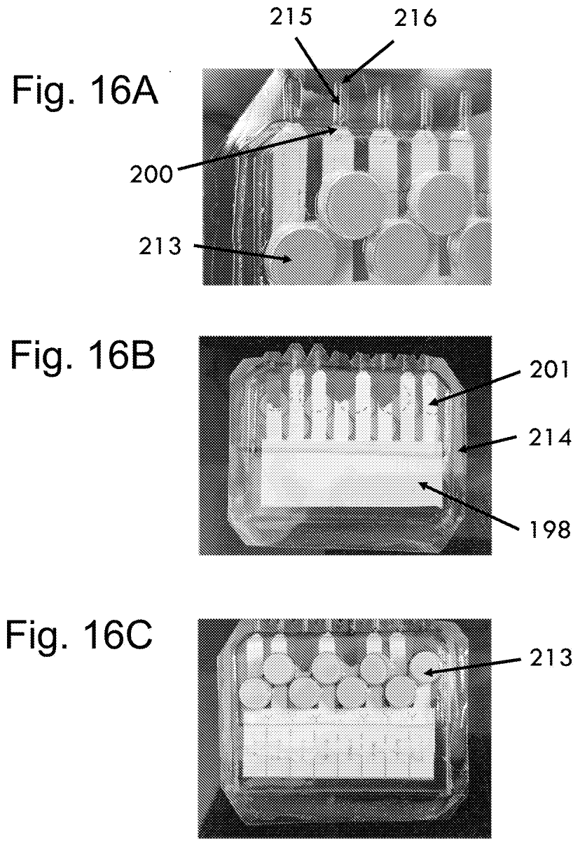

FIGS. 16A-16C shows the use of such packaged device. FIG. 16A shows one corner of a clear plastic thermoformed package containing a sample comb made of Whatman 903 paper having tines 201 whose tips 200 are located near the inner end of capillaries 215 which are initially closed off at the outer end 216, and desiccant tablets 213. In FIG. 16B (back view) and FIG. 16C (front view), the ends of several capillaries have been snipped off to open the channels to the outside, and blood has been applied to those channels, filling three of the tines. In this case the package was made of two sheets of vacuum-formed clear plastic material, each formed on a mold, and bonded together around the perimeter with glue. Such a 2-part plastic package is extremely common in retail packaging of many goods, and numerous methods can be used to generate such a package including vacuum forming, pressure forming, 3-D printing, injection molding, stamping, subtractive machining, etc. Numerous methods likewise exist to seal pieces of the package together including gluing, heat welding, ultrasonic welding, laser welding, etc. The embodiment shown can be used to collect a sample by snipping the tip 216 off of one of the capillary tubes 215 and applying a small droplet of blood to the end of the capillary, whereupon the blood fills the capillary and from there wets and is taken up by a tine of the sampling device. Further migration of humidity from the ambient atmosphere into the interior of the package can be prevented by formation of a plug in the capillary after the required amount of blood has flowed through and onto the sampling device tine. Such a plug can be formed by the drying of blood that remains in the capillary excess to the amount taken up by the device tine, by provision of a swellable material in the capillary that swells slowly once blood is introduced and blocks the capillary after the tine has been loaded, or by other means known in the art for the microfluidic control of biological samples in capillary channels. An 8-tine collection device packaged according to this embodiment can be used for collecting and drying 8 serial daily samples, or for any situation in which multiple discrete samples are required. The device is completely self-contained and can easily be carried with the user to collect samples at any time and place, and can be placed in an envelope and mailed to a centralized analytical laboratory where the package can be cut open and the comb device removed and processed as described above.

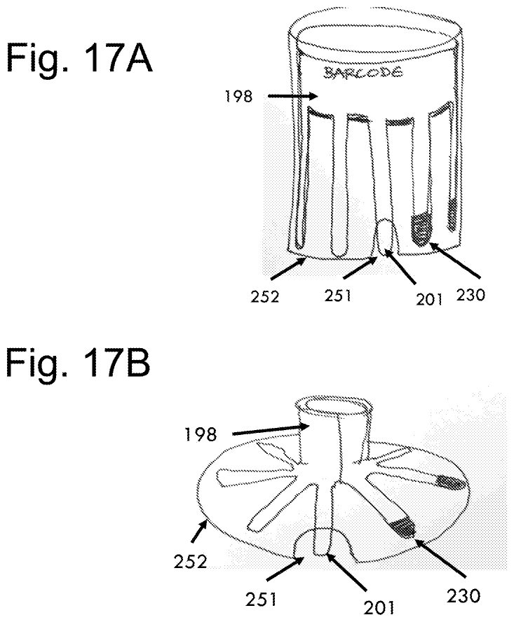

Alternative package embodiments shown in FIGS. 17A and 17B can provide isolation of a comb sampling device from the external atmosphere while allowing access to one tine at a time for sample application. FIG. 17A shows an embodiment in which the body 198 of a comb sampling device is curled into a cylinder and housed in a cylindrical shell container 252 having an opening 251 exposing tine 201 for sample loading. A previously loaded tine 230 has been moved away from the opening by rotation of the sample comb within the cylindrical housing. An alternative preferred embodiment is shown in FIG. 17B where the card 201 is bent into a cylindrical shape while the tines are splayed out radially. Tine 201 is accessible through a slot 251 in the housing 252, while previously applied sample 230 is shielded inside the housing by rotation of the card within the housing. In the two embodiments shown in FIGS. 17A and 17B, the housing can contain a desiccant capable of drying the samples within it (e.g., placed in the empty central space inside the cylindrical body of the device, or elsewhere within it). Opening 251 can be closed except when sample is being applied by providing a cap or sliding door mechanism, thus providing an airtight container suitable for controlled drying and storage of applied samples.

Estimation of Sample Amount

The amount of sample loaded on each tine of the comb device can be estimated by apportioning the total amount of sample that is loaded on the whole device into the individual (e.g., 8) samples based on the relative areas that the samples occupy on the material of the device. The total amount of sample on the comb device can be estimated based on the difference in weight of the device before and after samples are loaded (giving the weight of the samples themselves) and information on the water remaining in the sample at the humidity level to which they are equilibrated.

Alternative methods exist, or can be developed, to measure the amount of certain samples dried on the whole device or on individual tines. One such method is X-ray fluorescence (XRF) which can non-destructively measure several elements useful in the context of common biological samples. XRF measurement of iron (Fe) is particularly useful in the context of dried whole blood samples since there are precisely 4 atoms of iron in each tetrameric hemoglobin molecule. Thus a measurement of the amount of iron in the sample dried on a tine (or on the entire comb) can provide a direct estimate of the amount of hemoglobin. The amount of hemoglobin in a sample can be used to estimate the total amount of blood directly, and this estimate can be improved if the relative amounts of hemoglobin (contained in the blood's red cells) and albumin (present in blood plasma) are determined, for example by measurement of these proteins using the SISCAPA technology described elsewhere in this disclosure. XRF can also measure potassium (K) and sodium (Na), two elements present at very constant amounts in the blood of most individuals. Measurements of K and/or Na in a sample can therefore provide a direct estimate of the amount of blood in a sample by dividing the measured amount of K by the known typical concentration of K in blood, or similarly for Na. Parallel measurement of Fe as a surrogate for hemoglobin and red blood cells allows estimation of blood hematocrit through application of a measured calibration curve.

A valuable application of the measured dry sample weight (or equivalent weights determined as above) is to adjust the amounts of process reagents used in analysis of a sample to better preserve the desired stoichiometry with sample analytes. For example, in a SISCAPA protocol, it may be advantageous to adjust the amounts of disulfide reductants, alkylators, and trypsin to preserve a desired stoichiometric relationship with the amount of protein estimated to be in the sample. Such adjustments can be carried out under computer control using the sample dry weight to determine the volumes of reagents to be added to a sample well (or any other form of liquid vessel used om processing the sample) by a computer controlled pipetting system.

The relative amounts of sample solids and water measured by this approach allow normalization of analytical measurements to a consistent scale independent of variations in blood volume occurring in patients for reasons mentioned above.

Analytical Workflow

In a preferred embodiment related to protein analysis, blood samples dried on the tines of a comb device are placed in a 96-well plate as shown in FIG. 5B and prepared for digestion by dissolution, denaturation, reduction and alkylation. The purpose of such treatments is to dissolve the dried sample, open up the compact structures of the proteins, dissociate protein complexes and render each appropriate cleavage site (in the case of trypsin most lysines and arginines in the protein sequence) available to the proteolytic enzyme for cleavage. Since cystine intra- or inter-chain disulfide bonds play a major role in inhibiting protein unfolding, the reduction of cystine to two cysteines residues, and the modification of the resulting cysteines so as to prevent reformation of cystine bridges, are desirable steps in the sample preparation process. Briefly, each sample is subjected to dissolution by addition of liquid and shaking; dissociation (e.g., by addition of urea or deoxycholate); followed by cystine disulfide reduction (by addition, in minimal volumes, of dithiothreitol, mercaptoethanol or TCEP to a concentration of 2-4× the concentration of sample cysteine thiols, estimated at 26 mM in plasma before dilution), and, after incubation for 30 min (typically at 60° C. in the case of deoxycholate denaturation), alkylation of cysteines (by addition, in minimal volume, of iodoacetamide, iodoacetic acid, or the like, to a concentration 2× that of DTT just added) and incubation in the dark. Shaking the plate and included sample vessels significantly improves the rate and completeness of sample dissolution, particularly when an orbital shaking motion is used. In a preferred embodiment, dissolution, denaturation and reduction are carried out in one step, for example by addition of 280 μl of 2% deoxycholate, 1.7 μmol of TCEP in 0.25M Tris buffer pH 8.5 followed by vigorous shaking on an orbital plate shaker for 30 min at 60° C. Alkylation is then performed by addition of 3.4 μmol of iodoacetamide in water followed by incubation for 10 min the dark. Tryptic digestion is then carried out by addition of 360 μg of trypsin in 1 mM HCl, followed by shaking incubation at 40 C for 1 hour. Through this point, all steps can be carried out in a 96 well plate with the samples comb(s) in place, thereby avoiding any potential loss or fractionation of sample prior to analysis. After extraction of sample from the comb(s), the comb(s) can be removed from the vessel now containing the redissolved sample contents, preferably using an approach that reduces liquid remaining in the comb(s) (and thus removed from the sample used for analysis), for example by slowly lifting the comb(s) from the vessel so as to drain imbibed liquid into the vessel, or by lifting it above the sample vessel and centrifuging the vessel and comb(s) so as to move any remaining liquid from the comb(s) to the vessel. Removal of the comb(s) from a vessel holding extracted sample contents preferably occurs after addition of any internal standards used in subsequent analytical measurement processes so as to preserve the desired ratio between added standard and total amount of sample.

In a further preferred embodiment, the digested samples are subjected to SISCAPA enrichment of specific target peptides prior to mass spectrometric analysis. A preferred form of SISCAPA protocol employs anti-peptide antibodies immobilized on magnetic beads to capture target peptides and remove them from the digest. The magnetic bead capture may be carried out in the presence of the original sample vessel, after which the beads can be removed by magnetic capture (e.g., using a Tecan, Agilent, Hamilton or Beckman Coulter liquid handling robot), or the digest liquid may be transferred to a fresh vessel in which the SISCAPA capture, washing and elution steps are carried out so as to minimize any losses of magnetic beads adhering to or trapped within the sample absorber.

In a further preferred embodiment, the samples dried on tines of the device can be analyzed for certain classes of molecules using “paper-spray ionization” mass spectrometry (Wang, 2011) in which liquid and high voltage are applied directly to a strip of paper (here the tine) and a fine spray of sample molecules is generated from a point at the end of the strip and sucked into the inlet of a suitable mass spectrometer. The tips of the tines of the device may be formed as points for this application instead of the rounded as shown in FIGS. 1A-1C, thus improving the form of the electric field at the tip and thus of the spray.

In yet a further preferred embodiment, after samples are collected and dried on the tines of the device, a solvent is applied to the tip of the tines and allowed to migrate towards the body of the device. Using a solvent in which certain classes of sample molecules are soluble, but other classes of molecules insoluble, a chromatographic fractionation of the sample is carried out on the tine itself: if the tine is paper, this is equivalent to the classical technique of paper chromatography. The flow of liquid can be terminated after suitable fractionation is achieved and the soluble and insoluble molecules dried in their separated positions, after which they can be separately analyzed by excising the respective regions from the material of the tine. For example, if the solvent is a relatively hydrophobic (e.g., having a substantial proportion of organic solvent) lipophilic molecules will be transported along the tine towards the body of the device, whereas other molecules (e.g., large proteins) will remain where the sample was originally dried.

In yet a further preferred embodiment, an analytical workflow using dried samples collected using the device can include analysis of small molecules, for example by mass spectrometry. In general, small molecules are not affected by proteolytic digestion, and can thus be detected in eluted samples either before or after the sample is processed for other analytical targets,

In yet a further preferred embodiment, blood samples collected using the device can be analyzed using a workflow that includes extraction of native proteins including antibodies, and these antibodies can be tested to determine their specificities to understand what disease organisms the sample donor has previously been exposed to. For example, antibodies eluted from dried blood samples have been analyzed to show which subjects have generated antibodies to influenza virus, measles virus, or COVID19. This analysis can be carried by detecting antibodies to unique proteins from specific disease organisms and virus, or by detecting antibodies against peptide sequences specific to such an organism or virus. The elution of intact antibodies for such an analysis is compatible with subsequent use of the remainder of a dried blood sample for other tests including peptide analysis by mass spectrometry, which often involves proteolytic digestion of the remaining proteins to peptides. Most preferably, eluted intact antibodies can be used to capture specific peptides corresponding to antigen epitopes present on proteins produced by, or part of, an infectious organism or virus. Detection of such peptides from a viral protein as indicators of antibody against the virus, for example, can be carried out by several methods including mass spectrometry.

The use of mass spectrometry detection as a common component uniting detection of a subject's antibody repertoire, measurement of small molecules and measurement of specific endogenous proteins in the same sample allows these different types of tests to be carried out in a unified workflow using the same mass spectrometer detector. Combinations of two or all three of these methods provide greater simplicity and lower cost than separate assays, and enable development of instrument platforms with broad test menus for sample analysis including clinical applications.

Longitudinal Sample Collection

Sample collection books according to the invention can perform as a user's “dried blood sample diary”, enabling the user to collect samples with minimal additional equipment (e.g., lancets and optionally alcohol swabs and Band-Aids) anywhere and at any time.

In one embodiment, a user collects 8 samples on a daily basis, for a week plus one day. On the final day, the user collects two samples: the last tine of one card and the first tine of the next card, thus providing a sample overlap between the two 8-tine card sample collection devices. This overlap can be useful in confirming continuity of sample collection.

In a further embodiment, successive samples can be collected by a user at irregular times based on need or opportunity, with the circumstances of each sample recorded in the book or via a digital connection of some kind.

In a further embodiment, such a book can be used to collect samples from multiple individuals, with optional personal identification information. Such an approach can be useful for compact and rapid sample collection in population epidemiology studies.

It will also be clear to one skilled in the art that the comb devices and sample collection card formats described here can be used individually without assembly into a book or other type of container or covering. In one such embodiment, a rack of 12 sample collection cards, each having 8 tines, (approximating the arrangement shown in FIG. 5B) can be lowered into a 96 well plate containing 96 liquid samples, and all or a portion of each sample imbibed by the tines of the sample collection devices. After imbibing samples, the rack can be lifted free of the 96-well plate, creating a replica of the original set of 96 samples (in the manner by which microbiologists create “replica plates” of Petri dishes of colonies of microbes). This replica sample set can be further processed (e.g., for analysis), or the samples can be dried, cooled (e.g., frozen), or both, on the devices so as to preserve a replica sample collection. This approach to mass sample storage has the advantage that the replicated samples are ready for immediate placement in a 96-well plate to begin an analytical workflow without transfer to another container.

Use in Longitudinal Studies to Create Personal Baselines and Big Data

Many of the features of the invention facilitate the collection of longitudinal samples sets. Using the sample collection comb, typically in SampleDiary book format, together with an airtight box collection kit as disclosed here, a user can collect daily samples with minimal pain or inconvenience for months at a time, and incorporate regular sampling into a daily life routine. By making sample collection convenient, a routine involving daily collection proves be easier to maintain (i.e., provides higher user compliance) than collection at longer intervals that require scheduled reminders.

The extensive longitudinal data sets made possible by use of the invention enable discovery and development of a new class of diagnostic tests based on relative changes in one or more biomarkers over a specified time interval. For example, a test using two successive samples collected 1 day apart in which two biomarkers are measured could be interpreted as positive if the first biomarker decreases by 20% between time points while the second biomarker increases by 50%.

Likewise the data from many longitudinal samples from many subjects can be analyzed using both parametric and machine learning methods to reveal patterns over time associated with important medical outcomes.

Further embodiments include:
1. A sample collection device comprising a body and a plurality of tines, each tine having one or more sample application zones of absorbent material, where at least one of the sample application zones of each tine forms the end of the tine.
2. The device of 1, wherein at least two tines are parallel to each other.
3. The device of 1 or 2, wherein said absorbent material is porous and able to imbibe liquid samples by capillary forces.
4. The device of any of 1-3, wherein said absorbent material is a paper comprised of fibers, a non-woven synthetic material, a porous polymeric material or a patterned substrate.
5. The device of any of 1-4, wherein at least one of said sample application zones comprises a material that is different from the material of the body of the device
6. The device of any of 1-5, wherein the sample application zones are bonded or mechanically joined to the different material.
7. The device of any of 1-6, wherein said tines are uniformly spaced.
8. The device of any of 1-7, wherein said tines are uniformly spaced 4.5 mm or 9 mm apart.
9. The device of any of 1-8, wherein the number of tines is 8, 12, 16 or 24.
10. The device of any of 1-9, wherein the number of tines is greater than 100.
11. The device of any of 1-10, wherein said body of said device is identified by a unique computer-readable and/or human-readable code.
12. The device of any of 1-11, wherein at least one of said tines comprise a barrier preventing the flow of a liquid sample applied to a tip of said tines beyond a defined sample region.
13. The device of 12, wherein said barrier to sample flow is comprised of a solid material that occludes flow or a hydrophobic coating applied to the absorbent material of the device.
14. The device of any of 1-13, wherein the device comprises one or more reagents.
15. The device of 14, wherein the reagents are applied to, and dried on, regions of at least one of the tines before application of sample.
16. A sample collection booklet, comprising one or more sample collection devices of any of 1-15.
17. The sample collection booklet of 16, wherein said sample collection devices are bound between front and back covers
18. The sample collection booklet of any of 16-17, comprising two or more sample collection devices, and wherein said sample collection devices are separated by impermeable separator pages.
19. The sample collection booklet of any of 16-18, wherein said sample application zones of said sample collection devices are prevented from contacting covers or separator pages of said booklet by spacers.
20. The sample collection booklet of any of 16-19, wherein said sample collection devices are bound together by a ring-type binding.
21. A sample collection kit, comprising a sample collection booklet of any of 16-20, and a porous container of desiccant within an airtight reversibly closable container.
22. The sample collection kit of 21, further comprising a lancet.
23. The sample collection kit of 21 or 22, further comprising a digital device capable of measuring and recording or transmitting the relative humidity and/or temperature within said container.
24. A protocol for sample collection using the sample collection device of any of 1-15, the sample collection booklet of any of 16-20, or the sample collection kit of 21 any of 21-23, wherein a photograph of a newly collected sample is obtained and used to determine the date, time, or location of sample collection, or identity of a subject providing a sample.
25. The protocol of 24, wherein said photograph is captured by a mobile device and transmitted by electronic means.
26. The protocol of 24 or 25, wherein a user is prompted to collect a sample by a communication received through a mobile device.
27. An analytical workflow method, comprising analysing a sample collected using the sample collection device of any of 1-15, the sample collection booklet of any of 16-20, or the sample collection kit of any of 21-23 to determine a quantity of a biomolecule, metabolite, element or drug.
28. The workflow of 27, wherein the tines of a sample collection device are inserted into an array of vessels having the same spacing as the spacing between tines.
29. A database of measurements of biomolecules, metabolites, elements or drugs in samples collected using the sample collection device of any of 1-15, the sample collection booklet of any of 16-20, or the sample collection kit of any of 21-23.
30. The database of 29, wherein a multiplicity of said samples are collected at different times from a single individual and said measurements are used to compute a personal baseline value for one or more of said biomolecules or drugs.
31. The database of 29 or 30, wherein deviations of measurements from said personal baseline values by preset magnitudes are used to generate automatic communications to said individual.
32. A method, comprising contacting the sample device of any of 1-15 with a bodily fluid such that the bodily fluid is collected in one or more of the sample application zones.

1. A sample collection device, comprising a body and a plurality of tines, each tine having one or more sample application zones of absorbent material, where at least one of the sample application zones of each tine forms the end of the tine.

2. The device of claim 1, wherein at least two tines are parallel to each other.

3. The device of claim 1, wherein said absorbent material is porous and able to imbibe liquid samples by capillary forces.

4. The device of claim 1, wherein said absorbent material is a paper comprised of fibers, a non-woven synthetic material, a porous polymeric material or a patterned substrate.

5. The device of claim 1, wherein at least one of said sample application zones comprises a material that is different from the material of the body of the device.

6. The device of claim 5, wherein the sample application zones are bonded or mechanically joined to the different material.

7. The device of claim 1, wherein said tines are uniformly spaced.

8. The device of claim 1, wherein said tines are uniformly spaced 4.5 mm or 9 mm apart.

9. The device of claim 1, wherein the number of tines is 8, 12, 16 or 24.

10. The device of claim 1, wherein said body of said device is identified by a unique computer-readable and/or human-readable code.

11. The device of claim 1, wherein at least one of said tines comprise a barrier preventing the flow of a liquid sample applied to a tip of said tines beyond a defined sample region.

12. The device of claim 11, wherein said barrier to sample flow is comprised of a solid material that occludes flow or a hydrophobic coating applied to the absorbent material of the device.

13. The device of claim 1, wherein the device comprises one or more reagents.

14. The device of claim 13, wherein the reagents are applied to, and dried on, regions of at least one of the tines before application of sample.

15. A sample collection booklet, comprising one or more sample collection devices of claim 1.

16. The sample collection booklet of claim 15, wherein said sample collection devices are bound between front and back covers

17. The sample collection booklet of claim 15, comprising two or more sample collection devices, and wherein said sample collection devices are separated by impermeable separator pages.

18. The sample collection booklet of claim 15, wherein said sample application zones of said sample collection devices are prevented from contacting covers or separator pages of said booklet by spacers.

19. The sample collection booklet of claim 15, wherein said sample collection devices are bound together by a ring-type binding.

20. A sample collection kit, comprising a sample collection booklet of claim 15, and a porous container of desiccant within an airtight reversibly closable container.
